(19) United States
(12) Patent Application Publication
(10) Pub. No.: US 2024/0009248 A1
(43) Pub. Date: Jan. 11, 2024

(54) METHODS FOR GENERATING NEURAL PROGENITOR CELLS WITH A SPINAL CORD IDENTITY

(71) Applicant: University Health Network, Toronto (CA)

(72) Inventors: Michael G FEHLINGS, (US); Mohammad KHAZAEI, (US); Christopher S AHUJA, (US)

(73) Assignee: University Health Network, Toronto, ON (CA)

(21) Appl. No.: 18/044,288

(22) PCT Filed: Sep. 8, 2021
(86) PCT No.: PCT/CA2021/051239

§ 371 Date: Mar. 7, 2023

Related U.S. Application Data
(60) Provisional application No. 63/075,575, filed on Sep. 8, 2020.

(51) Int. Cl. A61K 35/30; C12N 5/0793; A61P 25/28

(52) U.S. Cl. CPC A61K 35/30; C12N 5/0619; A61P 25/28; C12N 2506/45; C12N 2501/11; C12N 2501/42; C12N 2501/115; C12N 2501/119; C12N 2501/415

(57) ABSTRACT

Provided herein are methods of producing spNPCs from iPSCs or NPCs, cell populations, compositions comprising cell populations, and uses of spNPCs made using the methods described. The method can comprise: a. obtaining unpatterned NPCs, the unpatterned NPCs expressing neuroectodermal markers including Pax6 and Sox1; b. priming the unpatterned NPCs of step a; and c. patterning the primed unpatterned NPCs to produce spNPCS.

### RELATED APPLICATIONS

This is a patent cooperation treaty application which claims the benefit of 35 U.S.C. § 119 based on the priority of U.S. Provisional Patent Application No. 63/075,575 filed Sep. 8, 2020, which is incorporated herein by reference in its entirety.

### FIELD

The present disclosure relates to methods of generating neural progenitor cells with a spinal cord identity from starter neural progenitor cells that express Sox2, Pax6, Nestin and at least one of the brain markers (such as Otx2, Foxg1 or Gbx2), or from induced pluripotent stem cells (iPSCs) or embryonic stem cells (ESCs), as well as to compositions comprising said cells or components for making said cells and uses thereof.

### INTRODUCTION

Spinal cord injuries (SCIs), frequently the result of falls or road traffic accidents, have devastating long-term physical, social, and financial impacts on patients and their families (Furlan et al., 2011; Sekhon and Fehlings, 2001). Mechanical compression and stretching of the spinal cord at the time of injury results in axonal damage as well as the loss of neurons and glia due to necrosis and apoptosis (Baptiste and Fehlings, 2006; Rowland et al., 2008; Tator et al., 1993). The loss of these critical cells prevents the transmission of neural signals from the brain to the rest of the body. Without these signals, an individual's ability to perform everyday activities such as walking, grasping and controlling bowel/bladder function is compromised. Cell transplantation with neural stem/progenitor cells to repair and regenerate the spinal cord is a promising treatment strategy for SCI (Khazaei et al., 2017; Nagoshi et al., 2018). A wide range of neural progenitor cells at different developmental stages have been studied as potential treatments. These studies have revealed multiple mechanisms underlying recovery, including cell replacement and trophic support. These mechanisms are believed to be partly determined by the source and developmental stage of the transplanted cells.

The generation of stem cells expressing distinct neural phenotypes is a complex multi-stage process, which starts during early neural tube development with invagination of the neuroectodermal layer to form neuroepithelial stem/progenitor cells (NPCs). NPCs in the rostral neural tube with an anterior identity ultimately form forebrain forebrain NPCs (fbNPC). Conversely, NPCs exposed to a temporally and spatially different gradient of morphogens gradually mature and caudalize, leading to the formation of the mid- and hind-brain. After further temporospatial maturation, these cells become further caudalized and then ventralized to form spinal cord NPCs (spNPCs)(Gifford et al., 2013). fbNPC and spNPCs are temporally and spatially located at different spectrums of this continuum (FIG. 1A), and their unique differentiation profiles likely contribute to differences in recovery observed following transplantation. Further, the fate of transplanted cells is affected by the microenvironment of the spinal cord injury site, where several cell fate determining factors, such as Shh, BMP, TGFβ and Notch, are upregulated(Chamankhah et al., 2013; Chen et al., 2005; De Biase et al., 2005).

Numerous central nervous system (CNS) disorders involve loss of key neuroglial cell populations. Cell-based regenerative approaches have therefore emerged as promising strategies to improve long-term outcomes for patients. Although early research employed primary donor cells, limited supply and the relative inaccessibility of CNS tissue made this approach infeasible for widescale therapy. As a result, the field has gravitated towards clinically relevant pluripotent stem cells (PSCs) as these cells can differentiate into any somatic human cell type.

Embryonic stem cells (ESCs) are PSCs capable of developing into the three primary germ cell layers in embryos. Studies of ESCs formed the basis for a substantial body of knowledge around stem cell culture and cell differentiation pathways. Unfortunately, limited availability and potential ethical concerns remain a challenge. The discovery of induced pluripotent stem cells (iPSCs) and associated culture protocols led to a resurgence of regenerative therapy research. iPSCs are reprogrammed cells derived from any somatic cell of any patient and are capable of forming all three germ cell layers as well as early-stage organoids. Their postnatal derivation eliminates ethical concerns and the potential to produce iPSCs as autologous therapies significantly reduces the risk of immune rejection.

### Neural Progenitor Cells

While numerous cell types, including mesenchymal stem cells, olfactory ensheathing cells and Schwann cells have been studied for central nervous system (CNS) regeneration¹, neural progenitor cells (NPCs), also known as neural stem cells, are the most promising as they are tripotent cells capable of differentiating into neurons, oligodendrocytes, or astrocytes; the primary cell types of the brain and spinal cord. To date, NPCs have been investigated for the treatment of numerous CNS conditions, including spinal cord injury (SCI), Parkinson's disease (PD), Alzheimer's disease (AD), amyotrophic lateral sclerosis (ALS), multiple sclerosis (MS), and stroke²⁻⁸.

### NPCs Demonstrate Different Regional Identities

The regional identity of brain and spinal cord NPCs is established early in neurodevelopment (FIG. 1,2). During embryogenesis, the neural tube is formed by infolding of the early ectodermal cells. These neuroectodermal cells in neural tube expressing the neural specific markers Sox2 and Pax6 become the earliest neural stem cells. These cells, also known as neuroepithelial cells. The early neural tube is a straight, elongated structure. Then the most anterior portion of the neural tube balloons into three primary regions, with the aid of transcription factors like Otx2, Lim1, and FoxA2, that form forebrain, midbrain and hindbrain. The posterior portion of the tube matures into spinal cord⁹.

The most anterior/rostral fate is the first regional identity that is established⁹. These cells express transcription factors such as Otx2, FoxG1, DIx2, Tbr1, and Tbr2^{10,11}. Foxg1, is expressed continuously in the postnatal and adult hippocampal dentate gyrus (DG)¹². Following rostral neural fate acquisition, the caudal identities will be determined by positional cues supplied by spatial gradients of patterning factors such as Wnt, FGFs, DKK1, and FRZB. These cells in the spinal cord express transcription factors, including HoxA2, HoxA3, and HoxB3¹³ (FIG. 2).

As in vivo, neural progenitors derived from PSCs in vitro first acquire rostral identity by default¹⁴, and this primitive identity can be converted to more caudal fates by various cues such as retinoic acid (RA), WNTs or FGFs , thereby mimicking the in vivo situation.

### Graft-Host Integration is Enhanced by Targeting the Regional NPC Identity

Unfortunately, a significant challenge remaining in NPC research is the optimization of graft-host integration to maximize functional recovery. Regional identity has emerged as a key consideration in the use of NPCs as therapy. Several studies have demonstrated that matching cell identity to the target tissue significantly improves graft-host integration and cell survival^{15,16}. For diseases predominantly in the spinal cord, such as SCI and ALS, this suggests a need to establish spinal cord-specific NPC lines. Classic protocols to derive pure NPC populations from PSCs generate brain-identity cells. Indeed, studies utilizing these brain identity NPCs found that they differentiate poorly into important populations such as spinal V2a interneurons and spinal motor neurons. Additionally, their integration is impaired by the introduction of foreign cortical populations, including excitatory pyramidal neurons, corticothalamic glutamatergic neurons, and cholinergic neurons. Regeneration of the corticospinal tract (CST) has also been observed following transplantation with adult spinal NPCs (spNPCs) but not forebrain NPCs (fbNPCs)¹⁷.

### The Need for a Human Spinal NPCs Protocol

The accumulating evidence in favour of regional-identity matching highlights a notable gap in current regenerative spinal research. Namely, most existing protocols to generate NPCs from human PSCs (hPSCs) produce cells expressing a forebrain and/or midbrain identity including transcription factors like Otx2, FogG1, Six3, and DIx2¹⁸⁻²¹. Developing a method of generating NPCs with a spinal identity is desirable and may improve cell integration and survival in the spinal cord niche.

### SUMMARY

Generation of human induced pluripotent stem cells (hiPSCs) from human peripheral blood or any somatic cells, provides a convenient and minimally-invasive method of obtaining patient-specific iPSCs. Described herein is a reproducible protocol for generation of spinal cord identity neural progenitor cells (spNPCs) from starter neural progenitor cells that express Sox2, Pax6, Nestin and at least one of the brain markers (such as Otx2, Foxg1 or Gbx2), or human pluripotent stem cells (hPSCs). The inventors have for example established a culture system and use a serum-free medium and a RAR agonist such as retinoic acid (RA), to differentiate iPSCs into neural progenitor cells (NPCs) with spinal cord identity.

spNPCs made using a method described herein can be characterized by immunocytochemistry staining and/or RT-qPCR analysis.

spNPCs can terminally differentiate into spinal cord specific neuronal cell types like ventral motor neurons and spinal interneurons, Renshaw cells, paragriseal, interstitial and propriospinal interneurons. These neuronal cell types cannot be generated by brain identity (e.g. unpatterned) NPCs. On the other hand brain NPCs can terminally differentiate to neuronal cell types of the brain like cortical, subcortical, or deep nuclear neurons, excitatory pyramidal neurons, Calbindin or CART expressing neurons, corticothalamic glutamatergic neurons and cortical cholinergic neurons that cannot be generated by spinal identity NPCs.

Starter neural progenitor cells (NPCs) that express Sox2, Pax6, Nestin and at least one of the brain markers (such as Otx2, Foxg1 or Gbx2), or PSCs such as hPSCs, hiPSCs and ESCs can be used after they have been differentiated to NPCs. hPSCs, which were used in Examples 1 and 2, expressed pluripotency-associated genes Oct4, Sox2, and Nanog before NPC differentiation. After differentiation, spNPCs expressed general NPC markers (Nestin, Sox2, and reduced Pax6) and also regional identity markers (e.g. Hox genes). It is demonstrated herein that spNPCs generated can be expanded in a neurosphere suspension system or monolayer cultures. Like other NPCs with other regional identities, spNPCs were able to differentiate into cells expressing cellular markers for neurons (e.g. Fox3 and/or β-III-tubulin), astrocytes (e.g. GFAP and/or S100b), and oligodendrocytes (e.g. O1 and/or O4 and/or Olig2 and/or Olig1). Starter NPCs such as unpatterned NPCs have a higher level of brain markers and lower level of spinal markers (e.g. Hox genes) than spinal identity NPCs. For example, spinal NPCs do not express detectable level of (by Immune-staining) of brain markers.

As shown further herein, whole-cell patch clamp recording revealed that neurons differentiated from fbNPCs and spNPCs, exhibited electrophysiological properties of neurons, including action potentials.

A first aspect of the invention comprises a method of producing spinal identity neural progenitor cells (spNPCs), the method comprising:
a. optionally incubating dissociated unpatterned neural progenitor cells (NPCs) in suitable culture media supplemented with, FGF2 agonist, a FGF8 agonist, optionally FGF8, to produce posteriorized NPCs expressing higher levels of at least one Hox gene. optionally HoxA4 and/or HoxA5, and lower levels of at least one of the brain markers Gbx2, Otx2 and FoxG1 compared to unpatterned NPCs;
b. passaging the posteriorized NPCs and incubating the posteriorized NPCs in culture media (e.g. NEM) supplemented with a RAR agonist, optionally retinoic acid (RA) or a RA synthetic analog, optionally EC23, and a Wnt signaling activator, optionally Wnt3a or BML-284 hydrochloride, to produce caudalized NPCs expressing reduced levels of one or more of Gbx2, Otx2 or FoxG1 levels compared to posteriorized NPCs;
c. passaging the caudalized NPCs in suitable culture media supplemented with a RAR agonist, optionally RA or a RA synthetic analog, optionally EC23; and
d. passaging the caudalized NPCs of step c) in suitable culture media supplemented with a FGF2 agonist, optionally FGF2 or SUN11602, an EGF receptor agonist, optionally EGF or NSC228155, and 740Y-P or a synthetic agonist of 740Y-P, until the identity of the NPCs are stabilized as spNPCs;
wherein, a ROCK inhibitor is optionally added to the culture media on day 1 after each or at least one passage. The method can be initiated with unpatterned NPCs or posteriorized NPCs.

When starting with unpatterned NPCs, the unpatterned NPCs can be primed.

A second aspect of the invention comprises a method of priming unpatterned NPCs to stay in an ectodermal cell fate, the method comprising:
a. obtaining unpatterned NPCs, the unpatterned NPCs expressing neuroectodermal markers including Pax6 and Sox1;
b. priming the unpatterned NPCs of step a, the method comprising:
   i. adding EGF-L7 agonist, preferably EGF-L7 to culture media comprising the unpatterned NPCs of step a; and
   ii. optionally adding a Notch signaling activator, optionally DLL4, to the culture media, to maintain the unpatterned NPCs in the ectodermal fate.

Another aspect of the invention comprises a method of producing spNPCs from unpatterned NPCs, the method comprising:
a. obtaining unpatterned NPCs, the unpatterned NPCs expressing neuroectodermal markers including Pax6 and Sox1;
b. priming the unpatterned NPCs of step a, the method comprising:
   i. adding EGF-L7 agonist, preferably EGF-L7 to culture media comprising the unpatterned NPCs of step a; and
   ii. optionally adding a Notch signaling activator, optionally DLL4, to the culture media, to maintain the unpatterned NPCs in an ectodermal fate; and
c. patterning the primed unpatterned NPCs to produce spNPCS, the method comprising:
   i. dissociating the primed unpatterned NPCs and incubating the primed unpatterned NPCs in suitable culture media supplemented with FGF2 agonist, a FGF8 agonist, optionally FGF8, to produce posteriorized NPCs expressing higher levels of at least one Hox gene, optionally HoxA4 and/or HoxA5, and lower levels of at least one of the brain markers including Gbx2, Otx2 and FoxG1 compared to unpatterned NPCs;
   ii. passaging the posteriorized NPCs and incubating said cells in suitable culture media supplemented with a RAR agonist, optionally RA or a RA synthetic analog, optionally EC23, and a Wnt signaling activator, optionally Wnt3a or BML-284 hydrochloride, to produce caudalized NPCs expressing reduced levels of Gbx2, Otx2 and FoxG1 levels compared to posteriorized NPCs;
   iii. passaging the caudalized NPCs and incubating the caudalized NPCs in suitable culture media supplemented with a RAR agonist, optionally RA or a RA synthetic analog, optionally EC23; and
   iv. passaging the caudalized NPCs of step iii) for 2 to 3 more passages in suitable culture media supplemented with a FGF2 agonist, optionally FGF2 or SUN11602, an EGF receptor agonist, optionally EGF or NSC228155, and 740Y-P or a synthetic agonist of 740Y-P, until the identity of the NPCs are stabilized as spNPCs;
wherein, a ROCK inhibitor is optionally added to the culture media on day 1 after each or at least one passage.

Another aspect of the invention comprises a method of producing spNPCs from induced pluripotent stem cells (iPSCs), the method comprising:
a. producing unpatterned NPCs from the iPSCs, the method comprising:
   i. passaging the iPSCs and incubating said cells in iPSC culture media for about 2 days, for example 36 h to 4 days, optionally wherein the iPSC culture media comprises a TGFβ inhibitor, FGF2 agonist, Wnt inhibitor, and BMP inhibitor;
   ii. culturing the iPSCs in iPSC culture media without FGF2 agonist, optionally without FGF2, for about 4 days, wherein a BMP inhibitor or dual SMAD inhibitors (for inhibition of both TGFβ and BMP pathways) is/are added to the culture media on about day 2, for example after about 36 h up to about 4 days;
   iii. culturing the iPSCs in NIM without an FGF2 agonist, optionally without FGF2, for about 2 days to produce embryoid bodies (EB); and
   iv. culturing the EBs of step iii in NIM with a FGF2 agonist, optionally FGF2 or SUN11602, for about 7 to about 11 days to produce neural rosettes, wherein the BMP inhibitor or dual SMAD inhibitors is/are removed from the media on about day 2, to produce unpatterned NPCs;
b. priming the unpatterned NPCs of step a, the method comprising:
   i. adding EGF-L7 agonist, preferably EGF-L7 to culture media comprising the unpatterned NPCs;.
   ii. optionally adding a Notch signaling activator , optionally DLL4, to the culture media, to maintain unpatterned NPCs in an ectodermal fate; and
c. patterning the primed unpatterned NPCs to produce spNPCS, the method comprising:
   i. dissociating the primed unpatterned NPCs and incubating the primed unpatterned NPCs in suitable culture media supplemented with a FGF2 agonist, optionally FGF2 or SUN11602, a FGF8 agonist, optionally FGF8, to produce posteriorized NPCs expressing higher levels of at least one Hox gene, optionally HoxA4 and/or HoxA5, and lower levels of at least one of the brain markers including Gbx2, Otx2 and FoxG1 compared to unpatterned NPCs;
   ii. passaging the posteriorized NPCs and incubating the posteriorized NPCs in suitable culture media supplemented with a RAR agonist, optionally retinoic acid (RA) or a RA synthetic analog, optionally EC23, and a Wnt signaling activator, optionally Wnt3a or BML-284 hydrochloride to produce caudalized NPCs expressing reduced levels of Gbx2, Otx2 and FoxG1 levels compared to posteriorized NPCs;
   iii. passaging the caudalized NPCs and incubating the caudalized NPCs in a suitable culture media supplemented with a RAR agonist, optionally RA or a RA synthetic analog, optionally EC23; and
   iv. passaging the caudalized NPCs of step iii) in culture media supplemented with B27, N2, FGF2 or SUN11602, EGF or NSC228155, and 740Y-P or a synthetic agonist of 740Y-P until the identity of the NPCs are stabilized as spNPCs;
wherein, a ROCK inhibitor is optionally added to the culture media on day 1 after each or at least one passage.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

An embodiment of the present disclosure will now be described in relation to the drawings in which:

FIG. 1 depicts a schematic showing neurons that are generated from neural stem progenitor cells with different regional identity, are restricted to differentiation to specific neuronal subtypes. Forebrain derived progenitors are not able to differentiate to spinal cord specific neuronal subtypes.

FIG. 2 depicts a schematic showing (left) key transcription factors found in the forebrain, midbrain, hindbrain, and spinal cord regions of the CNS. (Right) Select patterning morphogens driving development of different CNS regions during embryogenesis.

FIG. 3 depicts a schematic showing conceptual pathway to generate spinal NPCs from human PSCs. (Bottom) Temporal exposure of key patterning morphogens in vitro modeled on developmental cues.

FIG. 4 is an image depicting the morphology of neural rosettes (arrow heads) and neural tube-like structures (arrow).

FIG. 5A is a graph depicting gene expression profile of EGF-L7 primed NPCs (log2 fold change). FIG. 5B is a series of two images showing the morphology of primed NPCs (bottom) are similar to un-primed/unpatterned ones (top).

FIG. 6A is a graph showing qPCR-based Gene expression profile of posteriorized human NPCs versus un-pattern NPCs (log2 fold change). FIG. 6B is an image showing brightfield microscopy demonstrating the morphology of posteriorized NPCs.

FIG. 7A is a graph showing qPCR-based gene expression profile of caudalized human NPCs compared to posteriorized NPCs (log2 fold change). FIG. 7B is an image showing brightfield microscopy demonstrating the morphology of caudalized NPCs.

FIG. 8A is a graph showing qPCR-based gene expression profile of human spinal NPCs compared to caudalized NPCs and spinal NPCs (log2 fold change). FIG. 8B is an image showing the morphology of spinal NPCs on brightfield microscope demonstrating elongated processes and a less homogeneous appearance compared to conventional fore-brain NPCs.

FIG. 9A is a series of images showing a fore-brain neurosphere (left) and a spinal neurosphere (right). FIG. 9B is a graph showing the results of aneurosphere assay, which demonstrate the same self-renewal potential of fb-NPCs and spNPCs after 3 passages.

FIG. 10A is a series of images showing the comparison of the differentiation profile between fore-brain (top panels) and spinal human PSC-derived NPCs (bottom panels) expressing neuronal marker (βIII-tubulin). FIG. 10B is a series of images showing the comparison of the differentiation profile between fore-brain (top panels) and spinal human PSC-derived NPCs (bottom panels) expressing oligodendrocyte marker (O1). FIG. 100 is a series of images showing the comparison of the differentiation profile between fore-brain (top panels) and spinal human PSC-derived NPCs (bottom panels) expressing astrocyte marker (GFAP). FIG. 10D is a graph depicting the percent differentiation of fore-brain (top panels) and spinal human PSC-derived NPCs in cells expressing neuronal marker (βIII-tubulin), oligodendrocyte marker (O1), or astrocyte marker (GFAP).

FIG. 11A-C depicts voltage clamp recordings of spontaneous postsynaptic activity in fb-NPC derived neurons. FIG. 11D-F depicts voltage clamp recordings of spontaneous postsynaptic activity spNPCs derived neurons. The frequency and amplitude of postsynaptic events do not significantly differ between recordings in fb-NPCs and spNPC, indicating that much of the observed activity depends on synaptic transmission due to presynaptic action potentials.

FIG. 12 depicts: Generation and in vitro characterization of fbNPCs and spNPCs. FIG. 12A depicts a schematic showing spatial and temporal position of fbNPCs and spNPCs along the neural tube during nervous system development. FIG. 12B depicts a schematic showing fbNPCs are posteriorized, caudalized and ventralized to generate spNPCs. FIG. 12C depicts images showing the morphology of fbNPCs and spNPCs in culture (GFP+). FIG. 12D-E are graphs depicting quantitative real-time PCR analysis of the expression profile of fbNPCs and spNPCs relative to hiPSCs (D) and spNPCs relative to fbNPCs (E). Relative gene expression was determined by the 2-ΔΔ method and fold change values are relative to the mean gene expression in the housekeeping gene, GAPDH. (mean±SEM; *p<0.05, Student's t-test, n=3). FIG. 12F depicts a global view of RNA-seq analysis for differential gene expression (DEGs) between fbNPCs and spNPCs. Heat map depicting log10 scale of normalized TMP values (transcript pe million). FIG. 12G depicts a heatmap of unsupervised hierarchical clustering of significant enriched genes involved in neural tube pattern specification.

FIGS. 13A-B are graphs which depict a neurosphere forming assay for fbNPC and spNPCs, respecitively; cells were plated at a clonal density of 10 cells/μl. There was no difference in the number of neurospheres formed from fbNPC as compared to spNPCs in Naïve-h condition, but SCI-h resulted in formation of more and bigger fbNPC neurospheres. (mean±SEM; *p<0.05, Student's t-test, n=5). FIG. 13C are images depicting the neurospheres generated from fbNPC or spNPCs, when exposed to SCI-h. Scale bar: 50 μm. FIG. 13D depicts a graph illustrating the comparison of in vitro proliferation rate of fbNPC and spNPCs in different niches (Naïve-h vs SCI-h). At each time point, a BrdU proliferation assay was performed to determine cell number. Overall proliferation was comparable between fbNPC and spNPCs in the naïve niche. There were no significant differences in overall proliferation rate between the two groups, but in the SCI-niche the proliferation of fbNPC was much higher compared to spNPCs. (mean±SEM; **p<0.01 fbNPC compared to spNPC, two way ANOVA, n=3).

FIG. 14: In vitro Differentiation Assay of NPC lines. NPCs cultured and exposed to spinal cord homogenate from uninjured (Naïve-h) or SCI-lesioned animals (SCI-h). FIG. 14A are images depicting cells were fixed and stained for a neural progenitor cell marker (Nestin), oligodendrocyte marker (O1), astrocyte marker (GFAP), or neuronal marker (βIII-tubulin). Scale bar, 20 μm. FIG. 14B is a graph that depicts the percentage of cells positive for GFAP, O1, βIII-tubulin, or Nestin was quantified. (mean±SD; *p<0.05, **p<0.01, color matted to cell type, one way ANOVA, n=3). FIG. 14C depicts a graph an images showing the expression of DLL1, the Notch activating ligand, was increased in the tissue homogenate after spinal cord injury (SCI-h) at different time points. The fold change (log2) was calculated relative to naïve spinal cord homogenate (Naïve-h) and the loading control alpha tubulin. (mean±SEM; **p<0.01, one way ANOVA, n=3).

FIG. 15: Representative images characterization of the spinal cord lesion epicenter after transplantation of GFP+fbNPCs or spNPCs. FIG. 15A depicts images showing fbNPCs primarily migrated towards the lesion site to surround and partially fill the cavity, while spNPCs migrated along white matter both rostrally and caudally. FIG. 15B depicts images showing higher magnification images showing the migration of fbNPC into the cavity. fbNPCs fill most of the cavity space (left). spNPCs migrate rostral and caudal to the injury epicenter along white matter tracts (right). FIG. 15C is a graph showing quantitative analysis of transplanted cell distribution (mean±SEM; *p<0.05, two way ANOVA, n=3). FIG. 15D is a heatmap of unsupervised hierarchical clustering of significant enriched genes involved in neural tube pattern specification.

FIG. 16: hiPSC derived fbNPC and spNPCs demonstrate unique differentiation profiles within the chronically injured spinal cord. FIG. 16A is a series of images showing transplanted cells differentiate to express markers of undifferentiated NPCs (Nestin), mature oligodendrocytes (APC), immature oligodendrocytes (Olig2), astrocytes (GFAP) and neurons (Fox3) in spNPC and fbNPC groups. Scale bars: 20 μm. FIG. 16B is a series of graphs showing quantitative analysis of tri-lineage in vivo differentiation profiles. (mean±SEM; *p<0.05, Student's t-test, n=5).

FIG. 17: Transplanted spNPCs contribute to remyelination following SCI. FIG. 17A is a series of images showing generation of myelin is evident by colocalization of GFP+spNPCs and MBP in close apposition to endogenous NF200 positive axons (arrowheads). Scale bar: 20 μm. FIG. 17B is a series of images showing representative images of sagittal sections stained for Kv1.2 (arrowhead) and Caspr (arrow) in spNPC. Kv1.2+ juxtaparanodal voltage-gated potassium channel and Caspr+paranodal protein identified nodes of Ranvier. FIG. 17C is a series of images showing representative immunoelectron microscopic images from the GFP+spNPCs and fbNPC. Grafted cells were detected by the black dots observed upon GFP staining (black arrows). GFP+ black dots were often observed in the outer cytoplasm of the myelin sheath in spNPCs. However, in the fbNPC group, black dots are deposited inside the axoplasm, which is ensheathed by several layers of endogenous myelin. This indicates that graft derived neurons in the fbNPC group can be myelinated. Scale bar: 200 nm.

FIGS. 18A-B depict the results of an antibody array showing the different expression level of cytokines between fbNPC and spNPCs. The cytokine expression profile in the conditioned media collected from cells was detected using antibody array, which allows the detection of 41 cytokines and growth factors in one experiment. The fresh medium, without cell culture, was used as a background control. FIG. 18C depict the results of a histomorphometric analysis using LFB and H&E staining. Representative images of the spinal cord at the lesion epicenter and 0.96 mm rostral and caudal to the area in vehicle, spNPCs and fbNPC. FIG. 18D is a graph showing spatial quantification of the area of preserved white matter in different transplantation groups (mean±SEM, *p<0.05, **p<0.01, ***p<0.001, n=5). FIG. 18E is a graph showing spatial quantification of the area of lesional tissue in different transplantation groups (mean±SEM, *p<0.05, **p<0.01, ***p<0.001, n=5). FIG. 18F is a series of representative images of very high-resolution ultrasound (VHRUS) analyses for cavitation and FIG. 18G depicts a graphs showing quantitative analysis of cavity volume as assessed by VHRUS (mean±SEM, n=5, *p<0.01, *p<0.05, one-way ANOVA). FIG. 18H is a series of images showing the functional vascularity was measured using power Doppler VHRUS And FIG. 18I is a graph showing functional vascularity was measured using power Doppler VHRUS. No significant changes were observed between the grafted groups, although a trend toward improvement was observed in the fbNPC transplanted group. (mean±SEM; *p<0.05, **p<0.01, one way ANOVA, n=5).

FIG. 19: fbNPCs and spNPCs can make synaptic connections endogenous cells and are invovled in electric conduction. FIG. 19A is a series of images showing transmission electron micrographs of spinal cord sections at the site of transplantation demonstrating the formation of synapses between anti-GFP immunogold (black dots, white arrowheads)—labeled cells and endogenous axon terminals. FIG. 19B is a series of images showing Immune staining for Synapsin I (Syn1). Scale bar=10 μm. FIG. 19C is a schematic showing that to test if graft derived neurons are able to contribute to electrical transmission, we analyzed electrically evoked compound action potential (CAP) transmission across the injury site (C4 to T1). FIG. 19D is a graph showing CAP over time. traces represent the average of six animals per group. FIG. 19E is a graph showing. quantification of CAP amplitude 10 weeks after transplantation (means±SEM, n=6; *P<0.05, one-way ANOVA compared to vehicle). FIG. 19F is a graph showing CAP latency in sham, vehicle, fbNPC, and spNPC groups. FIG. 19G is a graph showing CAP velocity in sham, vehicle, fbNPC, and spNPC groups. The conduction velocity was calculated as the recording distance (10 mm) divided by latency (t) (means±SEM, n=6; one-way ANOVA, P<0.05, one-way ANOVA compared to vehicle).

FIG. 20: Functional analysis of the rats following cell transplantation. FIG. 20A is a graph showing forelimb motor function was assessed using a grip strength meter. Cervical spinal cord injury caused severe deficits in forelimb grip strength, which improved progressively during the 8 weeks after transplantation. Grip strength following fbNPC and spNPC transplantation was markedly improved compared with vehicle and reached a plateau later (mean±SEM, *p<0.05, two-way ANOVA, n=16 per group). FIG. 20B is a graph showing percentage of eaten pellets over time illustrating the assessment of the skilled forepaw use and motor functioning using Montoya staircase test (mean±SEM, non-significant, two-way ANOVA, n=10 per group). FIG. 20C is a series of graphs showing quantification of different forelimb gait parameters at 8 weeks post transplantation (mean±SEM, *p<0.05, **p<0.01 one-way ANOVA, n=12 per group).

FIG. 21A depicts a graph showing quantification of cell survival (mean±SEM; non significant, Student's t-test, n=5). FIG. 21B is a series of images showing representative images of colocalization of GFP+ transplanted cells with Ki67. Scale bars: 20 μm. Transplanted spNPCs and fbNPCs rarely colocalize with proliferative marker Ki67. FIG. 21C is a graph showing percentage of grafted cells expressing Ki67. Quantitative analysis showed that less than 4% of transplanted cells express Ki67. (mean±SEM; non-significant, Student's t-test, n=5). FIG. 21D is a series of representative images 140 days following transplantation in NOD/SCID mice. GFP positive cells of spNPC and fbNPCs dispersed in the tissue (white arrow), but there were no tumor formation in the H &E staining.

FIG. 22A is a graph showing inclined plane over time, illustrating global motor function was tested by each animal's ability to sustain increasing degrees of incline with platforms ranging from 0° to 90°. An animal's ability to tolerate larger incline angles is associated with better functional recovery. Cell transplanted animals performed better than vehicle treated animals, although it was not significant. (mean±SEM, non-significant, two-way ANOVA, n=16 per group). FIG. 22B is a graph showing evaluation of thermal allodynia in the tail-flick test. There was no significant difference during the time course. FIG. 22C is a graph showing the evaluation of mechanical allodynia in the von Frey test in the forepaw and FIG. 22D is a graph showing the evaluation of mechanical allodynia in the von Frey test in the hindpaw. There were no significant differences among the groups in the tail-flick and von Frey tests. (mean±SEM, one-way ANOVA, n=8 per group).

FIG. 23 is a series of images showing when the cells commit to a neuronal fate, the anterior brain and ventral spinal cord NPCs differentiate to less Ptf1a expressing cells than posterior brain and dorsal spinal cord NPCs. Ptf1a expressing cells will differentiate to inhibitory GABAergic interneurons.

FIG. 24 is a graph showing the percentage of eaten pellets over time in groups transplanted with posterior brain NPCs (posterior-NPC), dorsal spinal cord NPCs (dorsal-NPC), anterior brain (cNPC) and ventral spinal cord NPCs (spNPC). Transplantation of posterior brain and dorsal spinal cord NPCs resulted in less improvement in functional recovery than anterior brain and ventral spinal cord NPCs.

FIG. 25 is a series of images and a graph showing that there is no significant difference in the differentiation of cells toward oligodendrocytes (O1 positive cells) in baseline differentiation medium without adding any homogenate.

FIG. 26A is a heatmap showing differential expression of the top 2000 genes with the highest variation between fbNPCs and spNPCs. FIG. 26B depicts the results of a gene ontology enrichment of differential gene expression between fbNPCs and spNPC. FIG. 26C is a graph wherein the horizontal axis shows log2 TPM of genes in fbNPCs.

FIG. 27 is a graph that shows the percentage of eaten pellets over time in fbNPC, spNPC, and vehicle groups. Transplanted cells instantly start to express and secrete trophic factors, but differentiation and integration to neural network or myelination takes time.

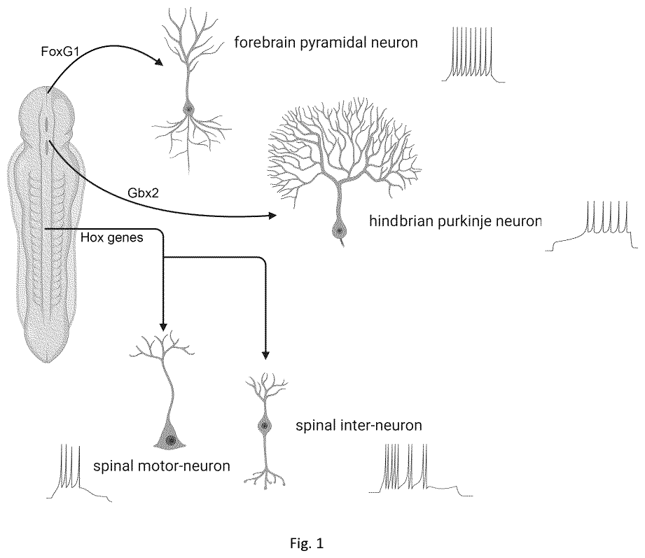

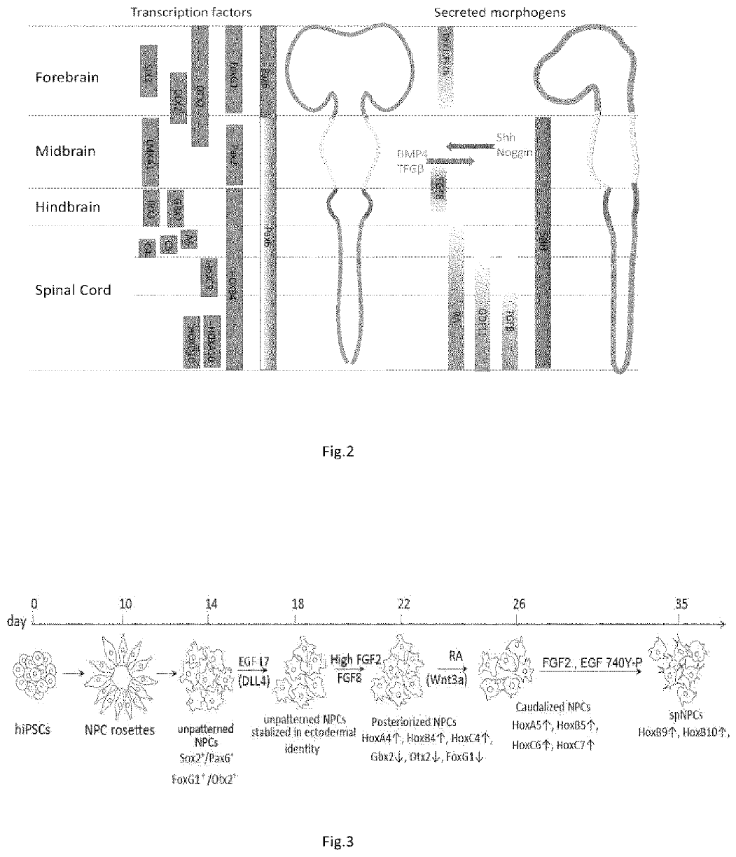

### DETAILED DESCRIPTION

Unless otherwise defined, scientific and technical terms used in connection with the present disclosure shall have the meanings that are commonly understood by those of ordinary skill in the art. Further, unless otherwise required by context, singular terms shall include pluralities and plural terms shall include the singular. For example, the term “a cell” includes a single cell as well as a plurality or population of cells. Generally, nomenclatures utilized in connection with, and techniques of, cell and tissue culture, molecular biology, and protein and oligonucleotide or polynucleotide chemistry and hybridization described herein are those well-known and commonly used in the art (see, e.g. Green and Sambrook, 2012).

As used in this specification and the appended claims, the singular forms “a”, “an” and “the” include plural references unless the content clearly dictates otherwise. Thus for example, a composition containing “a compound” includes a mixture of two or more compounds. It should also be noted that the term “or” is generally employed in its sense including “and/or” unless the content clearly dictates otherwise.

As used in this application and claim(s), the word “consisting” and its derivatives, are intended to be close ended terms that specify the presence of stated features, elements, components, groups, integers, and/or steps, and also exclude the presence of other unstated features, elements, components, groups, integers and/or steps.

The terms “about”, “substantially” and “approximately” as used herein mean a reasonable amount of deviation of the modified term such that the end result is not significantly changed. These terms of degree should be construed as including a deviation of at least ±5% or at least ±10% of the modified term if this deviation would not negate the meaning of the word it modifies.

As used herein, the term “pharmaceutically acceptable carrier” or variations thereof is intended to include any and all solvents, media, isotonic and absorption delaying agents, and the like, compatible with pharmaceutical administration and for use with cells. Optional examples of such carriers or diluents include, but are not limited to, buffered saline, culture media, Hanks' Balanced Salt solution, ringer's solutions, and 5% human serum albumin and bovine serum albumin (BSA).

The term “neural progenitor cell” or variations thereof also referred interchangeably as neural stem cell (NSC), neural precursor cells (NPC), neural stem progenitor cells (NSPCs), neuroepithelial stem/progenitor cells (NPC) or, neuroectodermal cells (NECs), as used herein includes neural cells that express Sox2, Pax6 and Nestin and are tripotent and differentiable to neurons, astrocytes or oligodendrocytes.

The term “neural progenitor cell with a spinal cord identity” or “spNPC” refers to neural progenitor cells that can terminally differentiate to spinal cord specific neuronal cell types like ventral motor neurons and spinal interneurons, Renshaw cells, paragriseal, interstitial and propriospinal interneuron cells, and which express elevated levels of spinal cord genes such Hox genes such as Hox A, B, C or D, 1-10 (e.g. A4, A5, B4, C4) in a higher amount than brain NPCs and express lower amounts of brain markers for example Gbx2, Otx2, FoxG1, Emx2 and/or Irx2 as well as Pax6 as compared to brain NPCs. Methods for producing spNPCs in vitro are provided herein.

Brain NPCs can differentiate to neuronal cell types of the brain like cortical, subcortical, or deep nuclear neurons, excitatory pyramidal neurons, Calbindin or CART expressing neurons, corticothalamic glutamatergic neurons and cortical cholinergic neurons that cannot be generated by spinal neurons.

The term “unpatterned NPCs” or variations thereof as used herein refers to NPCs that have a rostral identity, and are not caudalized and/or ventralized. Unpatterned NPCs are primitive or definitive NPCs which are not yet being treated with any patterning factors like RA or Shh (and its agonists). Unpatterned NPCs express Pax6, Nestin and Sox2 and at least one of the brain markers OTX2, FOXG1, and GBX2. The level of expression of Gbx2, Emx2 and Irx2 is lower in un-patterned NPCs as compared to mid-brain identity NPCs, and the level of expression of Hox genes (like A4, A5, B4, C4) are lower in un-patterned NPCs as compared to spinal cord identity NPCs. Unpatterned NPCs can be referred to as NPCs with forebrain identity (fbNPCs), NPCs with front brain identity, or anterior brain NPCs. All of these terms refer unpatterned NPCs. They can be used interchangeable throughout this disclosure.

The term “posteriorized NPCs” or variations thereof as used herein refers to tripotent neural progenitor cells with the same differentiation profile as unpatterned NPCs. The ability to form neurospheres and the proliferation rate of posteriorized NPCs are marginally higher than unpatterned NPCs. Treatment of unpatterned NPCs with a FGF8 agonist, optionally FGF8 under specific concentrations and time periods as described herein, results in posteriorization of the cells. Posteriorized NPCs express more Hox genes, such as HoxA4 and/or HoxA5, and have reduced expression of at least one of the brain markers such as DIx2, Six3, LmxA1, Gbx2, Otx2 and/or FoxG1 compared to unpatterned cells.

The term “EGF-L7 agonist” or variations thereof as used herein means EGF-L7, preferably human EGF-L7, (Accession number Ensembl:ENSG00000172889, MIM:608582) as well as active fragments, fusions and active splice variants thereof as well as any compound or combination of compounds, natural or synthetic, that simultaneously or in combination inhibits Notch signaling, activates EGFR (EGF receptor), inhibits ICAM-1 expression and enhances the inhibition of NF-κB activation. Combinations of molecules that activate a corresponding set of pathways can also be used instead of EGF-L7. For example a combination DLK1 or DAPT (to inhibit Notch), NSC228155 or betacelluin (to activate EGFR), A-205804 (to inhibit ICAM-1), and Bortezomib and Bardoxolone Methyl (to inhibit NF-KB) can be used. The term “EGF-L7” as used herein means EGF-L7, preferably human EGF-L7, (Accession number Ensembl:ENSG00000172889, MIM:608582) as well as active fragments, fusions and active splice variants thereof that simultaneously inhibits Notch signaling, activates EGFR (EGF receptor), inhibits ICAM-1 expression and enhances the inhibition of NF-KB activation.

The term “EGF receptor agonist” as used herein means any compound or combination of compounds, natural or synthetic that binds and/or induces EGF receptor (also known as EGFR; ErbB-1; HER1) tyrosine kinase activity, and includes without limitation EGF, and EGF analogs as well as heparin binding EGF (HB-EGF), transforming growth factor (TGF) amphiregulin (AR) and betacellulin. Also included are EGFR activators such as NSC228155.

The term “EGF” or variations thereof as used herein refers to epidermal growth factor. EGF, for example human EGF, includes active fragments, fusions and splice variants (e.g. fragments, fusions and splice variants that activate EGF receptor) can be obtained from various commercial sources such as Cell Sciences®, Canton, Mass., USA, Invitrogen Corporation products, Grand Island N.Y., USA, ProSpec-Tany TechnoGene Ltd. Rehovot, Israel, and Sigma, St Louis, Mo., USA.

The term “FGF2 agonist” or variations thereof as used herein means any compound or combination of compounds, natural or synthetic, that binds the FGF receptors that are bound by FGF2, such as FGFR1, FGFR2, FGFR3 and FGFR4, and includes FGF2, active fragments, fusions and splice variants thereof or molecules with similar function such as SUN11602 or combinations thereof.

The term “fibroblast growth factor 2” or “FGF2” or variations thereof (also known as bFGF, basicFGF, FGFb, or FGF-beta as well as heparin binding growth factor 2) as used herein refers to a member of the fibroblast growth factor family. FGF2, for example human FGF2, includes active fragments, fusions and splice variants(e.g. fragments, fusions and splice variants that activate FGF receptors that are bound by FGF2) can be obtained from various commercial sources such as Cell Sciences.RTM., Canton, Mass., USA, Invitrogen Corporation products, Grand Island N.Y., USA, ProSpec-Tany TechnoGene Ltd. Rehovot, Israel, and Sigma, St Louis, Mo., USA.

The term “FGF8 agonist” or variations thereof as used herein means any compound or combination of compounds, natural or synthetic, that binds the FGF receptors that are bound by FGF8, such as FGFR1, FGFR2, FGFR3 and FGFR4, and includes FGF8, active fragments, fusions and splice variants thereof or molecules with similar function such as FGF9, or FGF17 or active fragments, fusions and splice variants thereof (e.g. fragments, fusions and splice variants that activate FGF receptors that are bound by FGF8) as well as combinations thereof.

The term “FGF8” or variations thereof as used herein means FGF8 A, B or E isoform, referred to example as FGF8a, FGF8b or FGF8e and includes all naturally occurring or synthetic variants thereof, as well as mammalian FGF8 and in particular human FGF8, active fragments, fusions and splice variants thereof as well as combinations thereof. FGF8 is also called androgen-induced growth factor (AIGF).

The term “740Y-P”, “analog of 740Y-P” or variations thereof refer to a cell-permeable phosphopeptide activator of P13K RQIKIWFQNRRMKWKKSDGGYMDMS where Tyr-21 =pTyr) and analogs thereof and includes any compound or combination of compounds, natural or synthetic, that activates P13K kinase including for example erucic acid or active fragments, fusions and splice variants thereof as well as combinations thereof. 740Y-P can also be replaced with high doses of FGF2 or an FGF2 agonist, for example FGF2 at a concentration of greater than 20 ng/ml up to 400 ng/ml for example at least or about 200 ng/mL. 740Y-P is commercially available and can be purchased for example from Tocris Bioscience and Fisher Scientific.

The term “suitable culture media” as used herein means a culture media supportive of the particular cell type to be cultured. For example, a suitable culture media for neural progenitor cells or cells derived therefrom, such as PSC media, NEM or NIM, for example as described herein, comprising one or more additives_for example B27 or similar additive and optionally N2 or similar additive, appropriate for the stage of cells. Typically the culture media will include non essential amino acids such as Glycine, L-Alanine, L-Asparagine, L-Aspartic acid, L-Glutamic Acid, L-Proline, L-Serine, glucose or equivalent, sodium pyruvate, Catalase , Glutathione reduced, Insulin , Superoxide Dismutase, Holo-Transferrin , Triiodothyronine (T3), L-carnitine , Ethanolamine, D+-galactose, Putrescine, Sodium selenite, Corticosterone, Linoleic acid, Linolenic acid, Progesterone , Retinol acetate, DL-alpha tocopherol (vit E), DL-alpha tocopherol acetate , Oleic acid, Pipecolic acid, Biotin, optionally a FGF2 agonist such as FGF2 or SUN11602, EGFR agonist such as EGF or betacelluin, and optionally heparin are added.

The term “NEM” or “neural expansion media” or variations thereof as used herein means a base media suitable for culturing neural progenitor cells such as DMEM/F12, Neuralbasal Media etc comprising one or more of sodium pyruvate, a glutamine product such as glutamine or GlutaMAX™, one or more antibiotics such as penicillin and/or streptomycin, a supplement such as B27 supplement without vitamin A or equivalent (e.g. without RA or RA analog) and N2. Agonists can be added to the NEM depending on the stage of cell differentiation, for example one or more of an FGFR agonists such as FGF2, an EGFR agonist such as EGF and/or heparin. An example of a suitable NEM is provided in Example 1. Other suitable medias, supplements, antibiotics etc are known in the art and can be used.

The term “Notch agonist” or ““Notch signaling activator” or variations thereof as used herein includes any compound or combination of compounds, natural or synthetic including any small molecule or antibody that binds any Notch receptor and induces proteolytic cleavage and release of the Notch receptor intracellular domain. Examples include DLL1, DLL4, Jagged1, Jagged 2, including human and other mammalian versions thereof.

The term “Wnt agonist” or “Wnt signaling activator” or variations thereof as used herein includes any compound or combination of compounds, natural or synthetic including any small molecule or antibody that binds and activates a Wnt receptor. Examples include AZD2858, Wnt agonist 1, CP21R7 (CP21), Wnt, or BML-284 hydrochloride. Human and other mammalian versions of the biomolecule subset thereof are contemplated.

The term “Wnt inhibitor” or variations thereof as used herein includes any compound or combination of compounds, natural or synthetic including any small molecule or antibody that inhibits the Wnt signalling pathway. Examples include XAV939, DKK1, DKK-2, DKK-3, Dkk-4, POCN, C59, LGK-974, SFRP-1, SFRP- 2,SFRP-5,SFRP-3,SFRP-4,WIF-1, Soggy, IWP-2, IWR1, ICG-001, KY0211, Wnt-059, LGK974, 1WP-L6 and derivatives thereof as well as combinations thereof.

The term “RAR agonist” or variations thereof as used herein includes any compound or combination of compounds, natural or synthetic including any small molecule or antibody that binds and activates a RA receptor, including for example RA or an RA analog including for example EC23.

The term “B27” or variations thereof as used herein refers to a serum free vitamin containing supplement that supports neurons and which is used with neuronal cell culture. Any such supplement that permits feeder layer independent growth can be used. B27 supplement includes for example Catalase, Glutathione, Insulin, Superoxide Dismutase, Human Holo-Transferrin, T3, L-carnitine, Ethanolamine, D+-galactose, Putrescine, Sodium selenite, Corticosterone at, Linoleic acid, Linolenic acid, Progesterone at, Retinol acetate, DL-alpha tocopherol (vit E), DL-alpha tocopherol acetate, Oleic acid, Pipecolic acid-, and Biotin.

The term “rosette” or variations thereof as used herein refers to a cellular pattern of columnar cells. The express Sox1. The neural rosette is the developmental signature of neuroprogenitors in cultures of differentiating embryonic stem cells; rosettes are radial arrangements of columnar cells that express many of the proteins expressed in neuroepithelial cells in the neural tube. In addition to similar morphology, neuroprogenitors within neural rosettes can differentiate into the main classes of progeny of neuroepithelial cells in vivo: neurons, oligodendrocytes, and astrocytes.

The term “caudalized NPCs” or variations thereof as used herein refers to NPCs having a caudal spinal cord progenitor fate and which express Sox2, Pax6 and an increased expression of Nkx6.1 relative to un-patterned NPCs and a decreased expression of Six3, DIx2, Otx2 and FoxG1 relative to un-patterned NPCs. For example, “caudalized NPCs” express Sox2, Nestin and Pax6 with equivalent level to un-patterned NPCs, and have for example at least 75% decreased level of expression for FoxG1, Otx2 and Gbx2, at least 25% increased expression Nkx6.1, and have at least 25-50% increased expression of HoxA4, HoxB4 HoxC4 and HoxC5, all relative to un-patterned-NPCs. The expression level of Nkx6.1 is for example at least 25% less than the expression level this gene compared to ventralized-NPCs.

As used herein “neural induction media” or “NIM” or variations thereof herein means a base media suitable for culturing neural precursor cells such as DM EM/F12 comprising one or more of sodium pyruvate, a glutamine product such as glutamine or GlutaMAX™, one or more antibiotics such as penicillin and/or streptomycin, a supplement such as B27 supplement without vitamin A, non-essential amino acids such as Glycine, L-Alanine, L-Asparagine, L-Aspartic acid, L-Glutamic Acid, L-Proline, L-Serine, to which BMP inhibitor such as LDN193189 or Noggin, TGF8 inhibitor (such as SB431542), FGFR agonist such as FGF2, optionally heparin and EGFR agonist, optionally EGF. An example of a suitable NIM is provided in Example 1 in Table 1.

The term “pluripotent stem cell” or variations thereof as used herein refers to a cell with the capacity, under different conditions, to differentiate to more than one differentiated cell type, and for example the capacity to differentiate to cell types characteristic of the three germ cell layers, and includes embryonic stem cells and induced pluripotent stem cells, which are reprogrammed from somatic cells. Pluripotent cells are characterized by their ability to differentiate to more than one cell type using, for example, a nude mouse teratoma formation assay. Pluripotency is also evidenced by the expression of embryonic stem (ES) cell marker. Induced pluripotent stem cells (iPSCs) including human iPSCs (hiPSCs) can be from any somatic cell for example skin keratinocytes or fibroblasts and can be derived from a subject or a cell line. PSCs can for example be fetal derived, embryonic derived, or human embryonic stem cell derived.

The term “stem cell” or variations thereof as used herein, refers to an undifferentiated cell which is capable of proliferation, self-renewal and giving rise to more progenitor cells having the ability to generate a large number of mother cells that can in turn give rise to differentiated or differentiable daughter cells. The daughter cells can for example be induced to proliferate and produce progeny that subsequently differentiate into one or more mature cell types, while also retaining one or more cells with parental developmental potential.

The term “cell culture medium” or variations thereof (also referred to herein as a “culture medium” or “medium”) as referred to herein is a medium for culturing cells containing nutrients that maintain cell viability and support proliferation and optionally differentiation. The cell culture medium may contain any of the following in an appropriate combination: salt(s), buffer(s), amino acids, glucose or other sugar(s), antibiotics, serum or serum replacement, and other components such as peptide growth factors, vitamins etc. Cell culture media ordinarily used for particular cell types are known to those skilled in the art.

The term “passaging”, “passaged” or “passage” or variations thereof as used herein refers to transferring the cultured cells from their current growth medium to a new growth medium. Cells can be passaged for example according to as described in Example 1. Any suitable method of passaging however can be used. For example hiPSCs should be passaged in order to avoid overgrowth and to maintain them in an undifferentiated state. Further it may be preferable to passage iPSCs in clumps.

As a person skilled in the art would understand, cells can be dislodged from the culture plate with the use of enzymes and enzyme cell detachment solutions such as the enzyme cell detachment solution Accutase™. Other enzymes like Dispase, ReLeSR or TrypLE can also be used. In addition non-enzymatic methods, like EDTA solution, can also be used.

The term “N2” and non-commercial preparations thereof referred to as “hormone mix” refers to a hormone mix comprising transferrin, insulin, putrescine, selenium and prodesterone. For example N2 can comprise 10 mg/ml Transferrin, 2.5 mg/ml Insulin, 1 mg/ml Putrescine, 1 ul/ml 15 Selenium, 1 μl/ml Prodesterone. N2 can be purchased commercially from Gibco (Invitrogen/Themor scientific), Sigma and others or can be prepared.

The definitions and embodiments described in particular sections are intended to be applicable to other embodiments herein described for which they are suitable as would be understood by a person skilled in the art.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about”.

The inventors have identified improved methods for producing spinal identity neural progenitor cells (spNPCs) from unpatterned NPCs and intermediate cell populations along the path to spNPCs.

The method can comprise one or more features of Steps 2 and 3 in Example 1. The features can be in timing, order, composition and/or selection of factors.

For example, Example 1 demonstrates that suitable culture media supplemented with FGF2, EGF and 740Y-P or a synthetic agonist of 740Y-P is useful for promoting and stabilizing the identity of spinal NPCs from caudalized-NPCs. Example 1 also demonstrates determining an effective range for 740Y-P. Cells prepared according to such methods, as indicated in Example 2, improve functional recovery when transplanted.

Accordingly, a first aspect of the invention comprises a method of producing spinal identity neural progenitor cells (spNPCs), the method comprising:
a. optionally incubating dissociated unpatterned neural progenitor cells (NPCs), optionally primed NPCs, optionally using a cell detachment solution, in suitable culture media supplemented with FGF2 agonist and a FGF8 agonist, optionally FGF8, to produce posteriorized NPCs expressing higher levels of at least one Hox gene, preferably HoxA4 and/or HoxA5 and lower levels of at least one of the brain markers Gbx2, Otx2 and FoxG1 compared to unpatterned NPCs;
b. passaging the posteriorized NPCs and incubating the posteriorized NPCs in suitable culture media supplemented with a RAR agonist, optionally retinoic acid (RA) or a RA synthetic analog, optionally EC23, and optionally a Wnt signaling activator, optionally Wnt3a or BML-284 hydrochloride, to produce caudalized NPCs expressing a reduced level of Gbx2, Otx2 and/or FoxG1 levels compared to posteriorized NPCs;
c. passaging the caudalized NPCs in suitable culture media supplemented with a RAR agonist, optionally RA or a RA synthetic analog, optionally EC23; and
d. passaging the caudalized NPCs of step c) in suitable culture media supplemented with a FGF2 agonist, optionally FGF2 or SUN11602, an EGF receptor agonist, optionally EGF or NSC228155, and 740Y-P or a synthetic agonist of 740Y-P, until the identity of the NPCs are stabilized as spNPCs;
wherein, a ROCK inhibitor is optionally added to the culture media on day 1 after each or at least one passage.

As mentioned herein, the method can be initiated using unpatterned progenitor cells or later stage cells such as posteriorized NPCs.

The inventors have identified that Wnt signaling activator is able to improve the activity of RA. For example, the inventors have identified that when the media in caudalizaiton step (e.g. where the posteriorized NPCs are treated with RAR agonist) is supplemented with for example Wnt3a, the increase in the level expression of Hox genes, in for example HoxA4 and/or HoxA5, is elevated in caudalized cells as compare to posteriorized cells and also the decrease in the level of expression of Gbx2, Otx2 or FoxG1 genes is boosted in caudalized cells as compared to posteriorized cells.

Stabilized cells have fixed identity and cannot go for example go back to other developmental identities by themselves without additional treatment. Stabilized cell will be maintained with their current identity in the proliferation or maintenance culture media. For example if they maintain their identity and gene expression profile as described herein for at least 5, at least 6 or at least 10 passages, they are considered stabilized.

A Rock inhibitor can be added after the first day in culture after each passage or a subset of passages, for example at least one, and optionally for one or more steps. It can be removed or left in for subsequent days if added on day 1. It can be removed for example by refreshing the media on day 2 after passage.

Notch inhibition is necessary for keeping cells in ectodermal fate. Retinoic acid (RA) and its analogs can push cells out of an ectodermal fate. Preparing spinal identity neural progenitor cells (spNPCs) requires both notch inhibition and RAR activation for example provided by RA. The inventors have determined that EGFL-7 can balance these required signaling pathways. It has dual function, it inhibits Notch and at the same time does not have adverse effect on RA signaling.

Another aspect of the invention comprises method of priming unpatterned NPCs to stay in an ectodermal cell fate, the method comprising:
a. obtaining unpatterned NPCs, the unpatterned NPCs expressing neuroectodermal markers including Pax6 and Sox1;
b. priming the unpatterned NPCs of step a, the method comprising:
   i. adding EGF-L7 agonist, preferably EGF-L7 to culture media comprising the unpatterned NPCs of step a; and
   ii. optionally adding a Notch signaling activator, optionally DLL4, to the culture media, to maintain the unpatterned NPCs in the ectodermal fate.

The methods can be combined.

Accordingly another aspect of the invention comprises method of producing spNPCs from unpatterned NPCs, the method comprising:
a. obtaining unpatterned NPCs, the unpatterned NPCs expressing neuroectodermal markers including Pax6 and Sox1;
b. priming the unpatterned NPCs of step a, the method comprising:
   i. adding EGF-L7 agonist, preferably EGF-L7 to culture media comprising the unpatterned NPCs of step a; and
   ii. optionally adding a Notch signaling activator, optionally DLL4, to the culture media, to maintain the unpatterned NPCs in an ectodermal fate; and
c. patterning the primed unpatterned NPCs to produce spNPCS, the method comprising:
   i. dissociating the primed unpatterned NPCs, optionally using a cell detachment solution, and incubating the primed unpatterned NPCs in suitable culture media supplemented with FGF2 agonist, a FGF8 agonist, optionally FGF8, to produce posteriorized NPCs expressing higher levels of at least one Hox gene, optionally HoxA4 and/or HoxA5, and lower levels of at least one of the brain markers including Gbx2, Otx2 and FoxG1 compared to unpatterned NPCs;
   ii. passaging the posteriorized NPCs in suitable culture media supplemented a RAR agonist, optionally RA or a RA synthetic analog, optionally EC23, and a Wnt signaling activator, optionally Wnt3a or BML-284 hydrochloride, to produce caudalized NPCs expressing reduced levels of Gbx2, Otx2 and/or FoxG1 levels compared to posteriorized NPCs; iii. passaging the caudalized NPCs in suitable culture media supplemented with RAR
   agonist, optionally RA or a RA synthetic analog, optionally EC23; and
   iv. passaging the caudalized NPCs of step iii) for 2 to 3 more passages in suitable culture media supplemented with FGF2 or SUN11602, EGF or NSC228155, and 740Y-P or a synthetic agonist of 740Y-P until the identity of the NPCs are stabilized as spNPCs;
   wherein, a ROCK inhibitor is optionally added to the culture media on day 1 after each or at least one passage.

Another aspect of the invention comprises method of producing spNPCs from induced pluripotent stem cells (iPSCs), the method comprising:
a. producing unpatterned NPCs from the iPSCs, the method comprising:
   i. passaging the iPSCs in iPSC culture media for about 2 days, for example 36 h to 4 days, optionally wherein the iPSC culture media comprises a TGFβ inhibitor, FGF2 agonist, Wnt inhibitor, and BMP inhibitor;
   ii. culturing the iPSCs in iPSC culture media without a FGF2 agonist, optionally without FGF2, for about 4 days, wherein a BMP inhibitor or dual SMAD inhibitors (for inhibition of both TGFβ and BMP pathways) is/are added to the culture media on about day 2, for example after about 36 h up to about 4 days;
   iii. culturing the iPSCs in NIM without a FGF2 agonist, optionally without FGF2, for about 2 days to produce embryoid bodies (EB); and
   iv. culturing the EBs of step iii in NIM with a FGF2 agonist, optionally FGF2 or SUN11602, for about 7 to about 11 days to produce neural rosettes, wherein the BMP inhibitor or dual SMAD inhibitors is/are removed from the media on about day 2, to produce unpatterned NPCs;
b. priming the unpatterned NPCs of step a, the method comprising:
   i. adding EGF-L7 agonist, preferably EGF-L7 to culture media comprising the unpatterned NPCs in step a; and
   ii. optionally adding a Notch signaling activator, optionally DLL4, to the culture media, to maintain unpatterned NPCs in an ectodermal fate; and
c. patterning the primed unpatterned NPCs to produce spNPCS, the method comprising:
   i. dissociating the primed unpatterned NPCs, optionally using a cell detachment solution, and incubating the primed unpatterned NPCs in suitable culture media supplemented with a FGF2 agonist such as FGF2 or SUN11602, a FGF8 agonist, optionally FGF8, to produce posteriorized NPCs expressing higher levels of at least one Hox gene, optionally HoxA4 and/or HoxA5, and lower levels of at least one of the brain markers including Gbx2, Otx2 and FoxG1 compared to unpatterned NPCs;
   ii. passaging the posteriorized NPCs and incubating the posteriorized NPCs in suitable culture media supplemented with a RAR agonist, optionally RA or a RA synthetic analog, optionally EC23, and a Wnt signaling activator, optionally Wnt3a or BML-284 hydrochloride, to produce caudalized NPCs expressing reduced levels of Gbx2, Otx2 and/or FoxG1 levels compared to posteriorized NPCs; and
   iii. passaging the caudalized NPCs and incubating the caudalized NPCs in suitable culture media supplemented with RAR agonist, optionally RA or a RA synthetic analog, optionally EC23;
   iv. passaging the caudalized NPCs of step iii) in suitable culture media supplemented with a FGF2 agonist, optionally FGF2 or SUN11602, an EGF receptor agonist, optionally EGF or NSC228155, and 740Y-P or a synthetic agonist of 740Y-P, until the identity of the NPCs are stabilized as spNPCs;
   wherein, a ROCK inhibitor is optionally added to the culture media on day 1 after each or at least one passage. In some embodiments, the method for producing unpatterned NPCs further comprises additional steps as described in Example 1.

In some embodiments, the FGF2 agonist is FGF2, preferably human FGF2.

In some embodiments, the FGF8 agonist is FGF8, preferably human FGF8.

In some embodiments, the EGFR agonist is EGF.

In some embodiments, the RAR agonist is RA.

It is demonstrated in the Examples, that FGF8, EGF-L7 and/or 740Y-P in the steps described, are useful for obtaining spNPCs.

Embryoid bodies are three-dimensional aggregates of pluripotent stem cells and express pluripotent cell markers for example Klf4 and/or Oct4.

Neuroectodermal cells consists of cells derived from ectoderm. One of the prominent markers of these cells is Sox1

The FGF8 agonist, is optionally FGF8, preferably FGF8b although FGF8a, FGF8e or combinations thereof can also be used.

The inventors have found for example that starting from unpatterned NPCs which can be from any source, that the combination and order of factors, for example exposure to EGF-L7 followed by high concentration of FGF2 and/or FGF8, a short pulse of Wnt activation with RA, followed by RA alone and a step with 740-YP produces spNPCs.

Dual SMAD inhibition refers to inhibition of both BMP pathway and TGFβ pathways. This can be accomplished with “dual SMAD inhibitors” which refer to an inhibitor that inhibits both pathways or a combination of inhibitors that inhibit both the BMP pathway and TGFβ pathway, for example a BMP inhibitor and a TGFβ inhibitor. Examples for BMP inhibitors are: Noggin,

Dorsomorphin, LDN-193189, ML347, and LDN-212854 and DMH1. Others are also known for example SB431542, LDN-193189, PD169316, SB203580, LY364947, A77-01, A-83-01, GW788388, GW6604, SB-505124, lerdelimumab, metelimumab, GC-1008, AP-12009, AP-11014, LY550410, LY580276, LY364947, LY2109761, SB-505124, E-616452 (RepSox ALK inhibitor), SD-208, SMI6, NPC-30345, K126894, SB- 203580, SD-093, activin-M108A, P144, soluble TBR2-Fc, DMH-1, Dorsomorphin dihydrochloride and derivatives thereof as well as combinations thereof.

Examples for TGFβ inhibitors are: SB431532, PD169316, Galunisertib (LY2157299) or LY 3200882 as well as combinations thereof. Others are also known.

In another embodiment, the iPSCs are prepared from somatic cells from a mammal, such as a human. In an embodiment, the iPSCs are human iPSCs (hiPSC). In an embodiment, the iPSCs are prepared from a subject that has sustained a spinal injury. These cells can be used to prepare autologous spNPCs which can be used for example for autologous transplantation.

Various molecules can be substituted, for example, for BMP inhibition: LDN-193189, ML347, LDN-212854 and/or DMH1 can be used, for TGF-β. inhibition, PD 169316, Galunisertib (LY2157299) and/or LY 3200882 can be used, and for Wnt activation: KYA1797K, JW55 and/or POCN can be used.

In another embodiment, the unpatterned NPCs are incubated for about 3 days.

In another embodiment, the posteriorized NPCs are incubated for about an additional 3 days.

In another embodiment, the caudalized NPCs are incubated for about an additional 2 days.

In another embodiment, the concentration of FGF2 in the culture media used for producing posteriorized NPCs is from 20 ng/ml to about 150 ng/ml, for example, about 40 ng/mL.

Other FGF2 agonists can be used at a concentration that provides similar effect as FGF2.

In another embodiment, the concentration of FGF8 is from about 50 ng/ml to about 400 ng/ml for example about 200 ng/mL.

Other FGF8 agonists can be used at a concentration that provides similar effect as FGF8.

In another embodiment, the RA synthetic analog is about 0.1 μM EC23.

In another embodiment, the Wnt3a concentration is about 100 μg/ml. Other Wnt3a agonists can be used at a concentration that provides similar effect.

In another embodiment, the FGF2 agonist, optionally FGF2 or SUN11602, concentration in the culture media for passaging the NPCs until the identity of NPCs are stabilized is about 10 ng/ml.

In another embodiment, the EGF concentration is about 10 ng/ml.

Other EGFR agonists can be used at a concentration that provides similar effect as EGF.

In another embodiment, the 740Y-P concentration is about 1 μM.

In another embodiment, wherein the ROCK inhibitor is Y-27632.

In another embodiment, the concentration of the ROCK inhibitor is about 10 μM.

Other ROCK inhibitors can be used at a concentration that provide similar effect as Y-27632.

In another embodiment, the spNPCs passaged 3 to 10 passages.

In another embodiment, the concentration of EGF-L7 is about 10 ng/mL.

In another embodiment, the concentration of DLL4 is about 0.5 μM

Other Notch signaling activators can be used at concentrations that provide similar effect as DLL4.

Where an agonist, activator or inhibitor is a biomolecule such as a protein, mammalian and preferably human sequences are used.

In certain embodiments, the method further comprises enriching and/or isolating the desired cells.

Another aspect of the invention is a method comprises one or more steps as described in the examples.

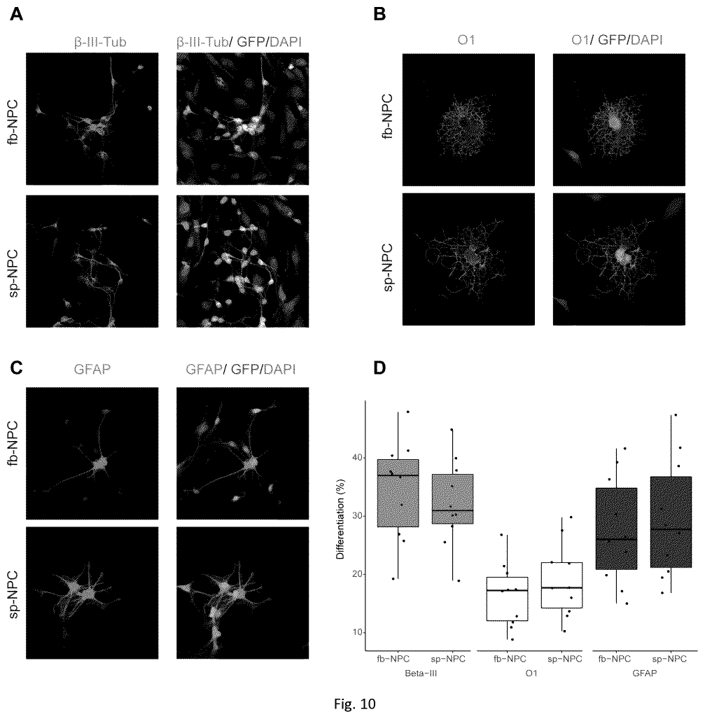

The spNPCs can also be further differentiated, for example, for neuronal differentiation, the spNPCs can be cultured in the absence of EGF and FGF but in the presence of BDNF, GDNF, Ascorbic acid and cAMP. As mentioned in Example 1, differentiated cells showed neuronal morphology and expressed the neuronal marker β-III-tubulin (FIG. 10). Astrocyte differentiation of spNPCs can be induced by exposure to BMP4 and CNTF, yielding cells with an astrocytic morphology that uniformly stained for glial fibrillary acidic protein (GFAP) (FIG. 10). Further spNPCs can differentiated towards an oligodendroglial fate, by for example sequentially applying a Shh agonist followed by PDGF-A. After 3 weeks of differentiation, staining revealed O1-positive cells with characteristic oligodendrocyte morphology.

Another aspect of the invention comprises a cell population comprising spNPCs or cells differentiated therefrom produced according to methods desbribed herein. The population of cells can be comprised in a composition, optionally in combination with a carrier, optionally a pharmaceutically acceptable carrier. In some embodiments, the population of cells are for use for transplantation in a recipient in need thereof. The pharmaceutically acceptable carrier can be a culture media or matrix, or freezing media, optionally GMP grade or sterile.

Another aspect of the invention comprises an isolated cell population of primed unpatterned NPCs comprising higher expression of Nest, Pax6 and Sox2 as compared to un-primed unpatterned NPCs. Another aspect of the invention comprises an isolated cell population of posteriorized NPCs comprising higher expression of HoxA4 and lower expression of Six3, DIx2, LmxA1, FoxG1, Gbx2, and/or Otx2 as compared to unpatterned NPCs. Another aspect of the invention comprises an isolated cell population of caudalized NPCs comprising higher expression of HoxA4 and lower expression of FoxG1, Gbx2, and Otx2 as compared to posteriorized NPCs. Another aspect of the invention comprises an isolated cell population of spinal NPCs comprising higher expression of HoxA4 and HoxA5 and less Gbx2 and Otx2 as compared to caudalized NPCs. The spNPCs described herein cannot be isolated or harvested in any quantity from tissue if at all. The methods described herein can be used to prepare for example autologous spNPCs, for example using fibroblasts or other somatic cells from a subject, such as a subject who has sustained a spinal injury, to prepare the isolated spNPCs.

In some embodiments, the higher or increased expression is at least 1 fold change (log2scale) higher/increased and/or the lower or reduced expression is at least 1 fold change (log2scale) lower/reduced.

Another aspect of the disclosure comprises an isolated cell population comprising spNPCs produced according to any of the methods described herein, optionally wherein the spNPCs are derived from a patient, optionally a spinal cord injury patient, optionally wherein the isolated cell population is for transplant, optionally autologous transplant.

Another aspect of the disclosure comprises a composition comprising any isolated cell population described herien and a pharmaceutically acceptable carrier, optionally a culture media or matrix, or freezing media, optionally GMP grade, xeno free media and/or sterile.

In some embodiments, any of the cell populations or compositions described herein are for use in treating a subject with a spinal cord injury or a neurodegenerative disease.

Another aspect of the disclosure is a method of treating a subject with a spinal cord injury or neurodegenerative disease or in the manufacture of a medicament for treating a subject with a spinal cord injury or neurodegenerative disease, the method comprising the administration of any of the isolated cell populations or the compositions described herein to treat a subject with a spinal cord injury or neurodegenerative disease or in the manufacture of a medicament for treating a subject with a spinal cord injury or neurodegenerative disease.

In some embodiments, the spinal injury is a cervical, thoracic or lumbar spinal cord injury, optionally acute or chronic.

In some embodiments, the neurodegenerative disease is multiple sclerosis (MS), Cerebral palsy (CP), Parkinson's disease, Alzheimer's disease, Huntington's disease, Amyotrophic lateral sclerosis, Friedreich's ataxia, Lewy body disease, spinal muscular atrophy, multiple system atrophy, dementia, schizophrenia, paralysis, multiple sclerosis, spinal cord injuries, brain injuries (e.g., stroke), cranical nerve disorders, peripheral sensory neuropathies, epilepsy, prion disorders, Creutzfeldt-Jakob disease, Alper's disease, cerebellar/spinocerebellar degeneration, Batten disease, corticobasal degeneration, Bell's palsy, Guillain-Barre Syndrome, Pick's disease, and/or autism.

Another aspect of the disclosure comprises a method of generating human neural stem/progenitor cells or neural precursor cell with spinal cord identity (spNPCs) comprising the following steps:
a) suspending pluripotent stem cells in a culture media containing a TGFβ inhibitor, FGF2 agonist, Wnt inhibitor, and BMP inhibitor;
b) subjecting the cells obtained in step (a) to suspension culture in a culture media containing a Wnt inhibitor and a BMP inhibitor;
c) forming an embryoid body by contacting the pluripotent human cell with an essentially serum free medium;
d) culturing the embryoid body to form rosettes and neural tube-like structure and neuroectodermal cells;
e) priming the neuroectodermal cells to stay in the ectodermal cell fate preferably by using EGF-L7 or its agonist(s) (for example a combination DLK1 or DAPT (to inhibit Notch), NSC228155 or betacelluin (to activate EGFR), A-205804 (to inhibit ICAM-1), and Bortezomib and Bardoxolone Methyl (to inhibit NF-κB)) and activating Notch signaling by DLL4 or any Notch signaling activating molecules,
f) posteriorizing the cells primed in e) in high concentration of FGF2 agonist, preferably FGF2, and FGF8 agonist, preferably FGF8, (for example greater than 20 ng/ml and up to about 400 ng/mL for FGF2 or FGF8);
g) caudalizing the cells posteriorized in f, preferably via treatment with RAR agonist, optionally any retinoic acid analog, and a Wnt agonist such as AZD2858, Wnt agonist 1, CP21R7 (CP21) or Wnt; and
h) induce proliferation capacity of spinal NPCs from the cells generated in step g) by dual activation of PI 3-kinase-Akt pathway and FGF pathway preferably using 740Y-P.

Another aspect of the disclosure comprises a cell culture composition for use in a step of deriving the spinal NPCs in vitro from pluripotent stem cells, wherein the spinal NPCs express one or more detectable markers for Sox2, Pax6, Nestin or vimentin, and the spinal NPCs have the capacity to differentiate into cells of a neural lineage, comprising a base cell culture composition optionally wherein the base cell culture composition for each step is as provided in Table 1 and further comprising EGF-L7 agonist, optionally EGF-L7, for priming the neuroectodermal cells to stay in the ectodermal cell fate as in claim 27e, FGF2 agonist, optionally FGF2 and/or a FGF8 agonist, optionally FGF8, for posteriorizing the primed cells as in claim 27e), RAR agonist, optionally RA, or Wnt3a for caudaulizing the posteriorized cells as in claim 27g, and/or 740Y-P for inducing proliferation capacity as in claim 27h, optionally in a concentration or concentration range as described herein. In some embodiments, the base media for each step is described in Table 1.

In some embodiments, the BMP inhibitor is selected from the group consisting of Noggin, Dorsomorphin, LDN-193189, ML347, and LDN-212854, DMH1 SB431542, LDN-193189, PD169316, SB203580, LY364947, A77-01, A-83-01, GW788388, GW6604, SB-505124, lerdelimumab, metelimumab, GC-1008, AP-12009, AP-11014, LY550410, LY580276, LY364947, LY2109761, SB-505124, E-616452 (RepSox ALK inhibitor), SD-208, SM16, NPC-30345, KÏ26894, SB-203580, SD-093, activin-M108A, P144, soluble TBR2-Fc, DMH-1, Dorsomorphin dihydrochloride and derivatives thereof as well as combinations thereof, preferably selected from Noggin, Dorsomorphin, LDN-193189, ML347, and LDN-212854, DMH1. In some embodiments, the BMP inhibitor is LDN193189.

In some embodiments, the dual SMAD inhibitors comprise a TGFβ inhibitor selected from the group consisting of SB431532, PD169316, Galunisertib (LY2157299) or LY 3200882 and a BMP inhibitor selected from Noggin, Dorsomorphin, LDN-193189, ML347, and LDN-212854, DMH1 SB431542, LDN-193189, PD169316, SB203580, LY364947, A77-01, A-83-01, GVV788388, GW6604, SB-505124, lerdelimumab, metelimumab, GC-1008, AP-12009, AP-11014, LY550410, LY580276, LY364947, LY2109761, SB-505124, E-616452 (RepSox ALK inhibitor), SD-208, SMI6, NPC-30345, KÏ26894, SB- 203580, SD-093, activin-M108A, P144, soluble TBR2-Fc, DMH-1, Dorsomorphin dihydrochloride and derivatives thereof as well as combinations thereof, preferably selected from Noggin, Dorsomorphin, LDN-193189, ML347, and LDN-212854, DMH1. Others are also known. In some embodiments, the TGFβ inhibitor is SB431542 or A-83-01.

In some embodiments, the Wnt inhibitor is selected from the group consisting of XAV939, DKK1, DKK-2, DKK-3, Dkk-4, POCN inhibitor, C59, LGK-974, SFRP-1, SFRP- 2,SFRP-Soggy, IWP-2, IWR1, ICG-001, KY0211, Wnt-059, LGK974, 1WP-L6 and derivatives thereof as well as combinations thereof. In other embodiments, the Wnt inhibitor is POCN. In further embodiments, the Wnt inhibitor is C59 or LGK-974.

In some embodiments, the pluripotent stem cell is a human pluripotent stem cell. In other embodiments, the human pluripotent stem cell is a human iPS cell or a human ES cell.

In some embodiments, the culture media further contains serum or a serum substitute. In other embodiments, the culture media comprises a ROCK inhibitor.

Another aspect of the disclosure comprises an isolated population of human pluripotent stem cell derived spinal neural stem/progenitor cells (spNPCs) produced according to any of the methods described herein, characterized in that the spinal neural stem cells express at least one of Nestin, Sox2, Pax6, optionally wherein the spNPCs comprise a phenotype similar to a conventional neural stem cell (NSC) and at least expression of one of the Hox genes (preferably Hox A4 or Hox A5). In some embodiments, at least one of the cultured cells expresses one of the detectable markers , HoxA4 or HoxA5 along with one or more detectable markers selected from Nestin, Sox2, and Pax6, wherein the amount of HoxA4 expression in the neural stem/progenitor cells is increased by at least 50% compared to the amount of HoxA4 expression in conventionally derived forebrain NPCs. In some embodiments, the cells are posteriorized cells and express more Hox genes, such as HoxA4 and/or HoxA5, and express less brain markers such as Gbx2, Otx2 and FoxG1 compared to unpatterned cells or fbNPCs. In some embodiments, the spNPCs differentiate to neurons, astrocytes or oligodendrocytes.

Another aspect of the disclosure comprises a composition comprising any of the isolated population of cells described herein and a carrier, optionally a pharmaceutically acceptable carrier, optionally a culture media or matrix, optionally GMP grade, xeno free media or sterile.

Another aspect of the disclosure comprises use of any of the population of spNPCs described herein or any composition described herein in the manufacture of a medicament for the treatment of a spinal cord injury of neurodegenerative disorder.

In some embodiments, the neurodegenerative disorder is multiple sclerosis, cerebral palsy, Parkinson's disease, Alzheimer's disease, Huntington's disease, Amyotrophic lateral sclerosis, Friedreich's ataxia, Lewy body disease, spinal muscular atrophy, multiple system atrophy, dementia, schizophrenia, paralysis, multiple sclerosis, spinal cord injuries, brain injuries (e.g., stroke), cranical nerve disorders, peripheral sensory neuropathies, epilepsy, prion disorders, Creutzfeldt-Jakob disease, Alper's disease, cerebellar/spinocerebellar degeneration, Batten disease, corticobasal degeneration, Bell's palsy, Guillain-Barre Syndrome, Pick's disease, and/or autism.

In some embodiments, the spNPCs are for transplantation into a brain or spinal cord of a patient.

Another aspect of the disclosure is a method of treating a spinal cord injury of neurodegenerative disorder, the method comprising the administration of any population of spNPCs described herein or any composition described herein to a patient in need thereof. In some embodiments, the spNPCs are for transplantation into a brain or spinal cord of a patient. In some embodiments, the neurodegenerative disorder is multiple sclerosis, cerebral palsy, Parkinson's disease, Alzheimer's disease, Huntington's disease, Amyotrophic lateral sclerosis, Friedreich's ataxia, Lewy body disease, spinal muscular atrophy, multiple system atrophy, dementia, schizophrenia, paralysis, multiple sclerosis, spinal cord injuries, brain injuries (e.g., stroke), cranical nerve disorders, peripheral sensory neuropathies, epilepsy, prion disorders, Creutzfeldt-Jakob disease, Alper's disease, cerebellar/spinocerebellar degeneration, Batten disease, corticobasal degeneration, Bell's palsy, Guillain-Barre Syndrome, Pick's disease, and/or autism.

Also provided is a composition comprising one or more of the cells generated using a method described herein, optionally in combination with a carrier.

As shown in the Examples, another aspect of the invention comprises a use of any cell population described herein or composition to treat a subject in need thereof, for example a subject with a spinal cord injury or neurodegenerative disease. The spinal injury may be a cervical or thoracic spinal cord injury, optionally acute or chronic. In an embodiment, the neurodegenerative disease is multiple sclerosis (MS), Cerebral palsy (CP), amyotrohic lateral sclerosis (ALS), Parkinson's disease (PD), Alzheimer's disease (AD), Huntington's disease (HD), Friedreich's ataxia, Lewy body disease, spinal muscular atrophy, multiple system atrophy, paralysis, brain injuries (e.g., stroke), cranical nerve disorders, peripheral sensory and neuropathies.

Also included in other aspect are uses of said cells and compositions comprising said cells for transplanting and/or treating a subject in need thereof, for example for transplanting and/or treating a subject with a spinal cord injury or degeneration, for example caused by a neurodegenerative disease, optionally MS or CP.

The cells or compositions may be combined with one or more neuroprotective factors, and/or the subject administered a cell population described herein may also be administered one or more neuroprotective factors, for example GDNF, BDNF, NT3, NGF, and/or CNTF. In one embodiment, the cells administered compris spNPCs or cells differentiated therefrom. In one embodiment, spNPCs which as shown may express more pro-oligodendrogenic factors, which helps remyelination, are administered wtih while fbNPC or other cell types.

Further, the definitions and embodiments described in particular sections are intended to be applicable to other embodiments herein described for which they are suitable as would be understood by a person skilled in the art. For example, in the following passages, different aspects of the disclosure are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1

An example of a step-by-step protocol for the generation of hPSC-NPCs with a spinal identity (spNPCs) starting from hPSCs is provided herein. The three main steps for the procedure: 1) Generation of unpatterned NPCs or embryoid body (EB) formation and dual-SMAD inhibition 2) Priming NPCs to ectodermal cell fate, and 3) Patterning of NPCs into spNPCs (FIG. 3).

Step 1: Generation of Unpatterned NPCs from hPSCs

Different methods of generating NPCs in vitro, including using “default pathway”^{22,23}, or via inhibition of SMAD signaling pathway. There are protocols utilising inhibition of just BMP pathway using a BMP inhibitor, or utilizing dual-SMAD inhibitors, to inhibit both TGFβ and BMP. The NPCs that are generated with these protocols in vitro, first acquire rostral identity by default¹⁴, before they get patterned to get other identities.

We use EB culture with dual-SMAD inhibition to generate NPCs with rostral identity. These cells are referred to herein as unpatterned NPCs.

If the hPSCs are cultured on a fibroblast feeder layer, they can be further expanded in feeder-free conditions for 3-4 passages prior to induction of neural progenitors. This action acclimates the cells, improving culture quality and yield.

To generate EBs, small clumps of hPSC will be cultured on ultra-low adherent dishes

in hPSC culture media (without FGF2) and neural induction media for 7 days. During this period, hPSCs grow to cell aggregates which are called EBs.

Neuroectodermal induction begins when EBs are transferred into the Neural Induction Medium (NIM) (around day 4-5). Plating EBs on Matrigel or Geltrex in NIM promotes the transition of cells into the rosettes with a neuroectodermal lineage that are expressing Sox1. Sox2 is also in hPSCs, but Sox1 starts after cells get neuroectodermal fate.

FGF2 signaling is necessary for the polarization of rosettes. Fibroblast growth factor 2 (FGF2) is then added to guide the transition of the neuroectodermal cells into rosette structures. NEM is for transitioning NPCs to produce NPC that express Nestin, Sox2, and Pax6 (e.g. unpatterned NPCs).

### Protocol:

1) Apply Accutase or ReLeSR (Stem Cell Technologies) as per manufacturer instructions to a healthy and homogeneous culture of hPSCs to separate them into cell clumps (consisting of 20-50 cells). Suspend the cells at a density of 1×10⁴ clumps/mL in hPSC culture media and add 2 mL to ultra-low adherent 6-well dishes. Incubate for two days under standard culture conditions of 37° C. and 5% CO₂ in a humidified incubator.

hPSC culture media (Table 1) without FGF is recommended.

TABLE 1
Composition of culture media

| | |
|---|---|
| Component | amount |

hPSC culture media

| DMEM/F-12 Medium | | |
|---|---|---|
| Knockout Serum Replacement (KOSR) | 20% | |
| L-Glutamine | 2 | mM |
| MEM Non-Essential Amino Acids | 1% | |
| FGF2* | 10 | ng/ml |
| 2-Mercaptoethanol | 0.1 | mM |
| Transferrin | 10 | μg/ml |
| IGF-I | 200 | ng/ml |

*at some steps FGF is not used
Neural Induction media (NIM)

| DMEM/F-12 and Neurobasal Media 1:1 | | |
|---|---|---|
| (supplemented with sodium pyruvate) | | |
| B27 minus vitamin A | 1x | |
| N2 supplement | 1x | |
| MEM Non-Essential Amino Acids | 1% | |
| FGF2* | 10 | ng/mL |
| heparin | 1.25 | U/L |
| TGFβ-inhibitor (SB 431542), | 10 | μM |
| BMP-inhibitor (Dorsomorphin) | 2 | μM |

*at some steps FGF is not used
Neural Expansion media (NEM)

| DMEM/F-12 and Neurobasal Media 1:1 | | |
|---|---|---|
| (supplemented with sodium pyruvate) | | |
| B27 minus vitamin A | 1x | |
| N2 supplement | 1x | |
| MEM Non-Essential Amino Acids | 1% | |
| FGF2 | 10 | ng/mL |
| EGF | 10 | ng/mL |
| heparin | 1.25 | U/L |

Alternate methods of passaging to Accutase dissociation include using 0.5 mM EDTA in Dulbecco's PBS without MgCl₂, CaCl₂, or ReLeSR. ReLeSR selectively lifts only iPSC cells, leaving differentiated cells on the plate. This allows for quick and easy selection for regular iPSC culture as well.

2) On day two, gently tilt plates to allow cell clumps to collect in the bottom corner of the well and replace half of the media from the top with fresh hPSC media supplemented with BMP inhibitor Dorsomorphin (2 μM) and TGFβ inhibitor SB431542(10 μM). Repeat this process on day 4.

Dorsomorphin and SB431532 block the BMP and TGF-β signaling pathways, which has been shown to improve the efficiency of neural induction to greater than 80% of total cells¹⁴.

3) On day 5, gently tilt plates and replace the media with NIM without FGF2 (Table 1).

Cell aggregates in the form of EBs should be observed by day 5. EBs simulate the endogenous conditions under which pluripotent hPSCs transition into neuroectodermal cells.

4) On day 7, transfer the EBs in NIM with FGF2 to a standard 6 cm culture plate pre-coated for 1 hour at 37° C. with Matrigel or Geltrex. Wait 24 hours before performing microscopy to confirm that EBs have completely settled and adhered to the plates.

5) On day 8, replace half of the media with fresh NIM with FGF2, but without Dorsomorphin. Repeat this process daily until day 13 to 17 (varies based on PSC cell line), at which point the first neural rosettes will form and then neural tube-like structures should be observed. The cells in rosettes or neural tube-like structures, unlike those in the periphery, should express early neuroectodermal markers such as Pax6 and Sox1.

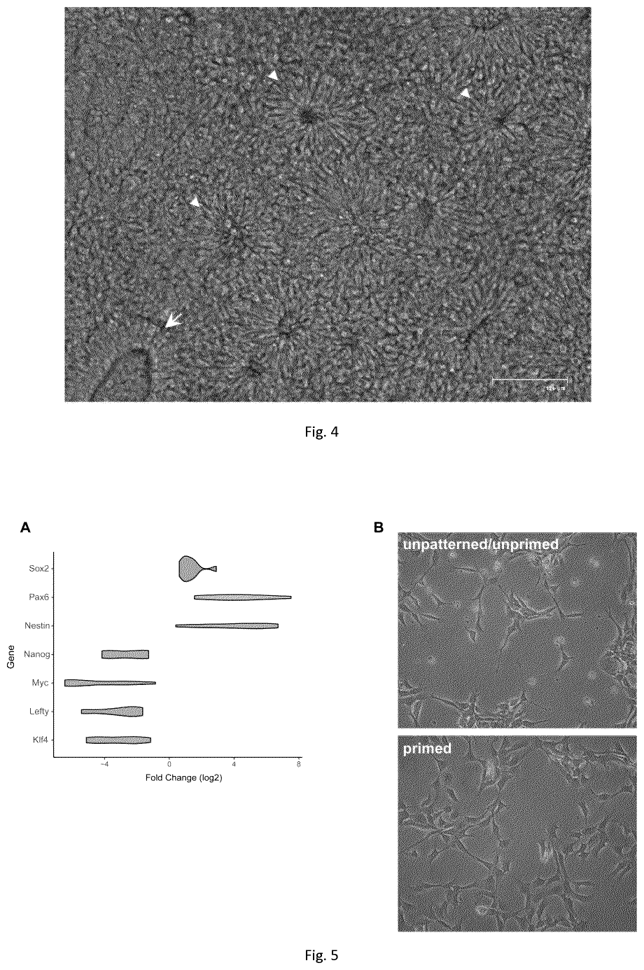

6) Two days after visualizing neural rosettes or neural tube-like structures, use a fine pipette tip to lift and transfer rosettes to a 15 mL Falcon tube containing NEM. Make sure to leave the non-neural cells at the periphery of the plate, as improper selection will impair NPC purity (FIG. 4).

Alternatively one can use either Neural Rosette Selection Reagent (Stem Cell Technologies), or a brief incubation (3-5 min) with Dispase, tapping, and a PBS wash to lift neural rosettes. Neural Rosette Selection Reagent had been found to be sub-optimal for selectively lifting neural rosettes of monolayer differentiation cultures, so use in only EB cultures is recommended.

7) Resuspend the selected rosettes at 1×10⁵ cells/mL in NEM and plate the cells onto PLL (0.1 mg/ml solution) and laminin pre-coated plates at 1×10⁵ cells/cm². Avoid re-plating at lower densities, as this promote undesired differentiation and loss of secondary rosette formation.

Laminin-511 (but not -332,-111, or -411) is preferred over other ECM replacements, such as Matrigel or Geltrex due to it being growth-factor free, which may interfere with the differentiation process.

After 7-8 days, lift the secondary neural rosettes manually (or with Dispase), and transfer them to another Matrigel-coated plate with N2B27 media. Following that, dissect the tertiary rosettes to purify NPCs.

Following re-plating, the culture should contain isolated NPCs that express Nestin, Pax6, and Sox2, but not Oct4.

### Clonal Expansion Neurosphere (Primary, Secondary, Tertiary).

8) Culture cells at 10 cells/μL on ultra-low adherent plates in NEM. Tilt plates and replace half the media with fresh media every 2-3 days for one week. At this stage, primary neurospheres should be observed as perfect spherical clusters of cells with a smooth contour and be at least 50 μm up to 150 μm in size. Neurospheres should not appear dark and ragged nor contain vacuoles or dead cells. They may also express Oct4, the marker for primitive NPCs.

9) Following detection, transfer neurospheres to 15 mL Falcon tubes with 500 μL of NEM. Use a flame-polished Pasteur pipette to pipette the media up and down 10-20 times, or until separation into single cells is observed. Plate the suspension at 10 cells/μL on ultra-low adherent plates in NEM.

### Expansion of Cells

10) Culture single cells at 1×10⁴ cells/cm² on PLUlaminin pre-coated standard culture plates in NEM containing 10 μM ROCK inhibitor. The next day, replace with fresh media containing NEM only.

11) After 5-6 days, use Accutase to passage cells to new PLL/laminin pre-coated plates with NEM. One day after passaging, supplement with 10 μM ROCK inhibitor.

Note: hPSC-NPCs generated using this method will, by default, express FoxG1, Gbx2 and Otx2, markers of forebrain to midbrain identity. Cells will not express HoxC4, a marker of spinal identity in NPCs.

### Step 2: Keeping the NPCs in the Ectodermal Cell Fate

During Step 1, Bone Morphogenetic Protein 4 (BMP4) signaling was inhibited by BMP inhibitor Dorsomorphin., LDN193189 (LDN) or Noggin can also be used, and TGFβ was inhibited by SB431542 (SB) to prevent mesodermal and endodermal differentiation. In the next step (Step 3) we are going to use Retinoic Acid (RA). RA tends to deviate differentiation of cells to a mesodermal fate²⁶.

To keep cells in the ectodermal fate, we need to inhibit Notch signaling when RA is active²⁷. It has been shown that inhibition of Notch signaling inhibits the differentiation to mesodermal fate and keep cells in the ectodermal layer²⁸. To inhibit notch signaling, we use the Notch antagonist EGF-L7 (10 ng/mL). EGF-L7 interacts with all the four Notch receptors (Notch1-4) and inhibits/competes with Jagged1 and Jagged2 proteins (not DLL4) for their interaction with Notch receptors²⁹. EGF-L7 knockdown stimulates the Notch pathway and EGF-L7 over-expression inhibits the Notch pathway. While NPCs are actively proliferating, Notch signaling contributes to the maintenance of the undifferentiated state.

Furthermore, by replacing the EGF in culture media with 10 ng/ml EGF-L7 in this step, EGF-L7 activates EGF-receptor, but it is less potent than EGF and modulates Notch signaling which reduce the hyper-proliferation of NPCs. Optionally we can also add 0.5 μM of DLL4 (DLL4: Delta-Like 4; a Notch signaling activator) with EGF-L7 to balance the reduction in Notch activity and keep the level of expression of neural progenitor genes like Nestin and Pax6 (FIG. 5).

There is some evidence that during development, unlike anterior neural progenitors, spinal progenitors can be also originated from neuromesodermal progenitors (NMPs). NMPs are able to differentiate into both paraxial mesodermal tissue and posterior neural tissue in vitro, and even further into specific neuron subpopulations such as motor neurons^{30, 31}. In vivo experiments in zebrafish have found that subpopulations of NMPs become fate restricted and spatially segregated, as well as having large differences in self-renewal potential³².

### Step 3: Patterning NPCs Towards a Spinal Cord-Specific Identity:

To generate spNPCs, we patterned cells using a stepwise treatment of morphogens³³. Patterning of primed NPCs towards a spinal cord identity is modelled on the developmental cues that are involved in the formation of the spinal cord during embryogenesis.

Early in differentiation, all neural cells have a rostral identity. However, future caudal cells gradually become posteriorized and then get the characteristics of the caudal midbrain and spinal cord. FGFs, Wnt, retinoic acid (RA), and Shh are involved in this spinal cord specification and subsequent elongation. Among RA, Wnt, and FGF signals, RA causes the strongest level of caudalization: inducing suppression of forebrain differentiation and promotion of caudal CNS specification.

The region of the neural plate giving rise to the spinal cord is specified in an FGF-dependent manner. Several FGFs, including FGF3, FGF4, FGF8, FGF13, FGF18, are involved in spinal cord specification. In vitro experiments have shown that exposure of neural tissue to increasing FGF levels results in progressively elevated levels of HOXC6, HOXC8, HOXC9, or HOXC10^{34,35}. Furthermore, several signaling pathways influence FGF8 expression. The Wnt and Shh pathways, which are active in the caudal region of the neural tube, can themselves increase FGF8 levels^{36,37}.

1) Dissociate cells into single cells using Accutase. Plate cells at 1×10⁴ cells/cm² on PLL/laminin pre-coated standard culture plates in DMEM:F12 media supplemented with B27, N2, FGF2 (40 ng/ml), FGF8 (200 ng/ml). Incubate under standard conditions for three days³³.

Table 2 contains a list of material that can be used for this protocol.

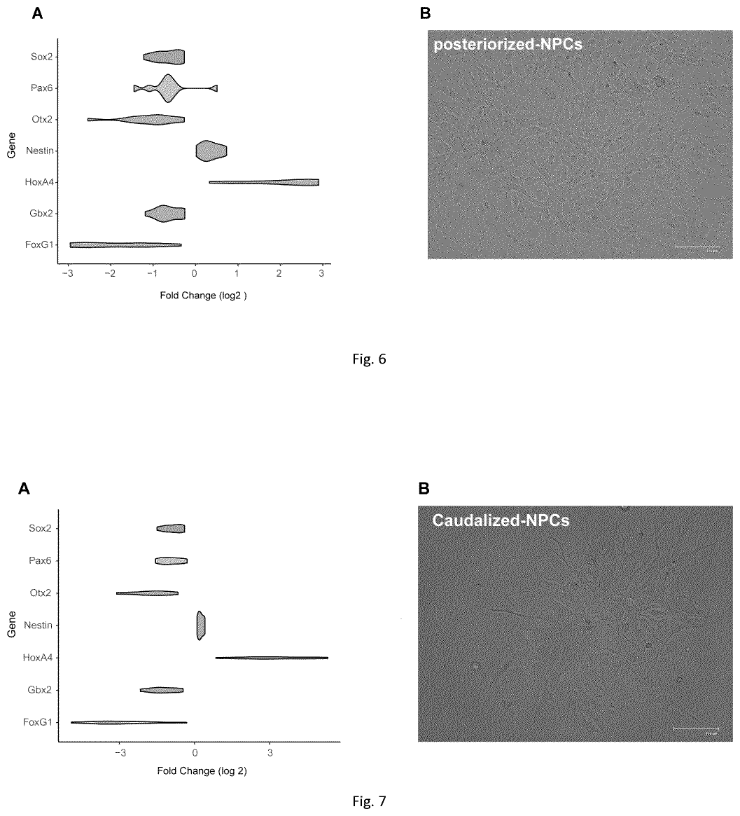

In this step we are using at least 2× more FGF2 (from 20 ng/ml to 150 ng/ml) and a high concentration of FGF8 (from 50 ng/ml to 400 ng/ml). In the embryo, caudal cells are exposed to select FGFs for longer periods of time than rostral cells they are involved in regionalization of the spinal cord along the rostral-caudal axis. During later stages of spinal cord elongation, FGF8 is more broadly expressed. Expression of FGF8 continues for several days but declines toward the final stages of somitogenesis and the cessation of axis elongation^{38, 39}. Treatment with FGF8 at this concentration and time period results in posteriorization of the cells. The posteriorized NPCs produced at the end of this stage express more Hox genes, such as HoxA4, and have reduced expression of at least one of the brain markers such as Gbx2, Otx2 and FoxG1 compared to un-patterned cells (FIG. 6). Posteriorized NPCs are equally tripotent with the same differentiation profile as un-patterned NPCs. The ability to form neurospheres and the proliferation rate of posteriorized NPCs are marginally higher than un-patterned NPCs.

2) On day 3, use Accutase to passage cells to new PLL/laminin pre-coated standard culture plates in DMEM:F12 media supplemented with B27, N2, 0.1 μM EC23, and Wnt3a (100 μg/ml). Incubate for an additional 3 days.

In this step we induce caudalization of cells using retinoic acid (RA) or the synthetic retinoid analog, EC23. Using EC23 is preferred as it is more photostable at incubation temperatures.

FGF and RA signaling are not sufficient (alone or together) to induce caudal characteristics in neural cells grown in vitro and Wnt signaling (Wnt3a) is further required to specify neural cells to a caudal identity⁴².

3) On day 6, use Accutase to passage cells to new PLL/laminin pre-coated standard culture plates in DMEM:F12 media supplemented with B27, N2 and 0.1 μM EC23. Incubate for an additional 2 days.

No Wnt3a is required at this stage.

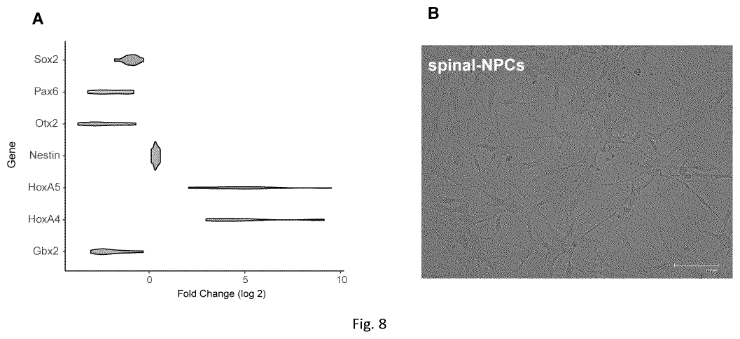

Treatment with RA and Wnt for 3 days results in caudalization of cells. These caudalized NPCs express Hox genes such as HoxA4. EC23 is continued for an additional 2 days after passaging to stabilize the caudal identity of the NPCs. This additional RA pathway activation results in a significant reduction (to nearly no expression) of Gbx2, Otx2 and FoxG1 levels compared to posteriorized cells (FIG. 7). Caudalized NPCs are also tripotent with the same differentiation profile as primed NPCs. However, the ability of caudalized NPCs to form neurospheres and their proliferation rate are significantly reduced compared un-patterned NPCs (FIG. 7).

4) Passage Caudalized-NPCs for 2-3 passages in DMEM:F12 supplemented with B27, N2, FGF2 (10 ng/ml), EGF (10 ng/ml) and 740Y-P (1 μM) until the identity of cells get stabilized, at this stage spinal NPCs are formed. Spinal NPCs can be passed and maintained in this media for up 3-5 more passages for optimum results, but passing up to P10 (and beyond) can be acceptable depending on the culture conditions (FIG. 8).

during maintenance period, the proliferation rate of cells is reduced. To generate

sufficient numbers of cells, prolonged culture for several passages is required. The concentration of FGF2 cannot be increased at this stage. To overcome this problem, 740Y-P is added which is as effective as FGF2 at promoting neuronal cell survival and proliferation via PI 3-kinase-Akt pathway⁴³. The effect of 740Y-P is dose dependent.

We recommend using spNPCs between ˜P3-P10. However, later passage cells may develop NPCs with mixed identity and cells that generate more GABA-ergic interneurons

After each passage, add 10 μM Rock inhibitor (Y-27632) on day 1 of culture.

TABLE 2
Materials

6-well Ultra-low Adherence Plates
740Y-P
Accutase
Dorsomorphin
Epidermal growth factor -like domain-containing protein 7 (EGFL-7)
Fibroblast Growth Factor-2 (FGF2)
Fibroblast Growth Factor-8b (FGF-8b)
Heparin
hPSC culture (hESCs or hiPSCs) (table1)
mTeSR Plus*
Laminin-511
Matrigel
Geltrex*
Neural Expansion media (NEM) (Table 1)
Neural Induction media (NIM) (Table 1)
Poly-L-lysine (PLL) 70-150 KDa
Recombinant Murine Wnt-3a
ReLeSR
Retinoic acid agonist EC23
ROCK inhibitor (Y-27632)
SB431542
Sonic hedgehog (Shh) agonist Purmorphamine

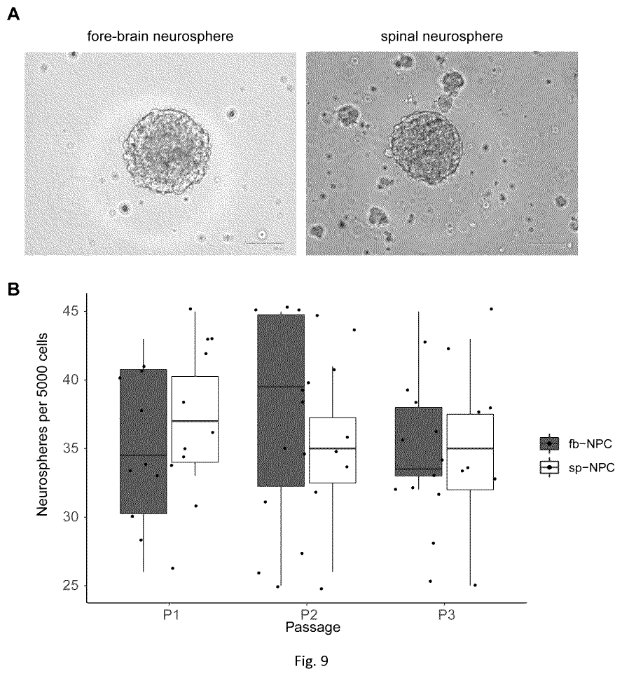

The self-renewal capability spNPCs is comparable to that of fbNPCs, as it was determined using a clonal analysis (FIG. 9). Neurospheres were mechanically dissociated to a single cell suspension and plated at clonal density in the neurosphere assay (FIG. 9). No significant difference was found in the self-renewal ability of fbNPCs or spNPCs during 3 passages.

The developmental potential of spNPCs are comparablee to that of fbNPCs as assesses by analysing their capacity for differentiation into the three main neural lineages. For neuronal differentiation, NPCs were cultured in the absence of EGF and FGF but in the presence of BDNF, GDNF, Ascorbic acid and cAMP. Differentiated cells showed neuronal morphology and expressed the neuronal marker β-III-tubulin (FIG. 10). Astrocyte differentiation of fbNPCs and spNPCs was induced by exposure to BMP4 and CNTF, yielding cells with an astrocytic morphology that uniformly stained for glial fibrillary acidic protein (GFAP) (FIG. 10). To compare the ability of fbNPCs and spNPCs to differentiate towards an oligodendroglial fate, we employed a protocol that sequentially applies a Shh agonist followed by PDGF-A. After 3 weeks of differentiation, staining revealed O1-positive cells with characteristic oligodendrocyte morphology.

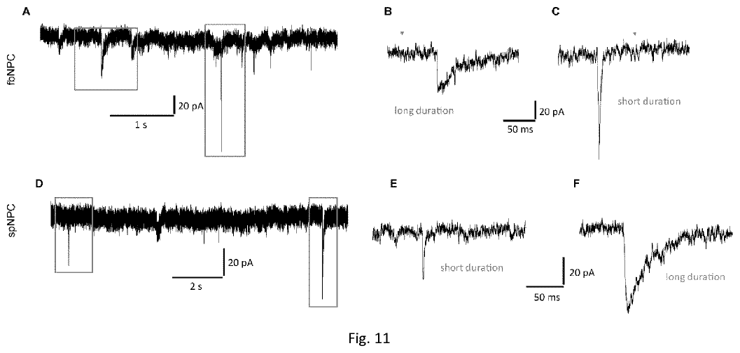

The spontaneous synaptic activity of neurons derived from spNPCs is comparable to that of fbNPCs as determined using whole-cell patch-clamp recordings that demonstrated both lines had similar inward sodium currents and were able to generate action potentials. Furthermore, the amplitude of sodium currents and the firing properties of neurons were not significantly different between the two groups (FIG. 11).

Multifaceted insults to the spinal cord after trauma or due to degenerative conditions can induce lasting sensorimotor deficits which hinder a patient's function. These impairments are largely due to tissue loss making targeted cell replacement a promising therapeutic strategy⁴⁴. The successful generation of spNPCs from iPSCs marks a significant milestone in developing effective cell-based treatments for patients where none currently exist. With the unique ability to match the niche of the target tissue, spNPCs have demonstrated improved integration and survival when transplanted into the spinal cord^{15,16} Of particular interest is their ability to regrow the CST and V2a circuit interneurons, which are crucial in restoring lost motor function^{15,16}. In contrast, conventional brain NPCs differentiate poorly into these populations and also produce undesired cortical cells which hinders their potential to produce meaningful outcomes. Together, these findings underscore the importance of matching the cell identity to the affected region to maximize regrowth and recovery.

The shift from forebrain-NPCs to spNPCs marks a key step in the optimization of cell-based therapies for degenerative spinal conditions.

Neurodegeneration and the subsequent lack of regeneration pose a major obstacle to the functional recovery of spine patients. Stem cell therapy is poised to remove this obstacle, with recent advances in cell programming moving the treatment one step closer to clinical application. Specifically, assigning iPSC-NPCs with spinal identity allows improved integration, repopulating the site of damage with the appropriate cells without introducing foreign cells originating from outside the spinal cord.

### Example 2

This study seeks to determine how the differentiation state of transplanted neural stem/progenitor cells is modulated by the spinal microenvironment to promote recovery. Using a rodent model of cervical spinal cord injury, NPCs at different stages of development (fbNPC vs spNPCs) were transplanted into a rodent model of cervical spinal cord injury and the mechanism(s) of recovery were examined.

Transplantation of tripotent neural stem/progenitor cells is a promising therapeutic strategy for traumatic spinal cord injury (SCI), however, the optimal temporal and spatial developmental stage for transplanted cells remains to be determined. In this study, we compared the fate determination of neuroepithelial stem/progenitor cells (NPCs) with an anterior forebrain identity (fbNPC) to NPCs patterned to acquire a ventral spinal cord identity (spNPCs) in the injured spinal cord microenvironment both makes Glutamatergic or GABAergic interneurons. Human induced Pluripotent Stem Cell (hiPSC) derived fbNPC and spNPCs were generated and transplanted into the injured spinal cord. fbNPC mainly differentiated into neurons, while spNPCs mainly differentiated to myelinating oligodendrocytes. The unique differentiation profiles were mainly due to differential Pax6 expression between the two lines, and were affected by activation of Notch signaling in the injured spinal cord microenvironment. Transplantation of both NPCNPCS lines resulted in neurobehavioral recovery, including improvements in forelimb grip strength and measures of forelimb/hindlimb locomotion, as assessed by Catwalk. fbNPC produced their effects in functional recovery through differentiation to neurons, which migrated towards the cavity and formed a cellular bridge. However, spNPCs produced their effects through remyelination. Both lines provided trophic support for tissue preservation and regeneration.

### Results

Characterization of in vitro Generated Cells

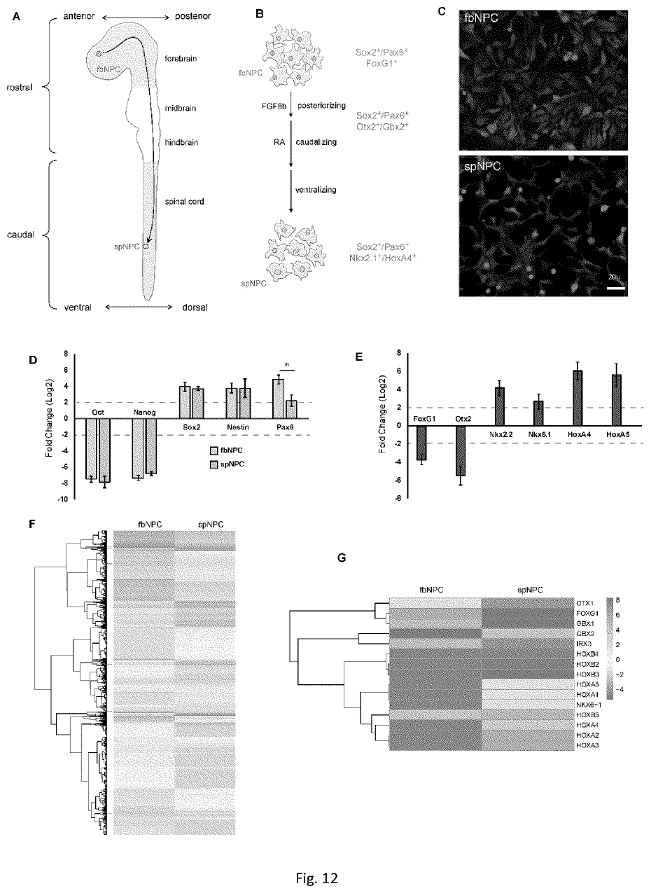

GFP-positive-hiPSCs, generated by non-viral, piggyBac transposon-mediated reprogramming (Hussein et al., 2011), were used to establish fbNPC and spNPCs by mimicking key morphogenic cues and replicating developmental neural tube patterning in vitro (FIGS. 12A-C). A combination of different growth and patterning factors were applied to induce the generation of fbNPC and spNPCs. For the establishment of fbNPC, a dual SMAD inhibition method was used(Varga et al.). At the earliest stages after generation of fbNPC, they were maintained in a culture media to preserve the anterior identity of cells (forebrain identity) (Payne et al., 2018; Varga et al.). spNPCs were generated using dual SMAD inhibition, caudalized and ventralized using agonists for retinoic acid (RA) and Sonic hedgehog (Shh), and maintained in a culture media to preserve their ventral spinal cord identity using the method described in Example 1.

A comparative gene expression analysis revealed that the expression of pluripotent cell markers (Oct4, Nanog) was decreased, whereas expression of neural cell markers (Sox2, Pax6 and Nestin) was increased in both lines compared to the original hiPSCs. The expression of Nestin and Sox2 was comparable between fbNPC and spNPCs (FIG. 12D), but fbNPC were observed to express 2.2 fold more Pax6, than spNPCs (FIG. 12D). Gene expression profiling of fbNPC showed higher expression levels of transcription factors Otx2 and FoxG1, which are markers of anterior identity cells, as compared to spNPCs. Conversely, spNPCs expressed increased levels of Nkx2.2, Nkx6.1, HoxA4 and HoxA5 transcription factors, which represent spinal cord identity. (FIG. 12E). To compare the global transcriptome of fbNPCs and spNPCs, we performed RNA-seq analysis (FIG. 12F). Despite the considerable similarity of gene expression patterns in fbNPCs and spNPCs, we identified some important differences. There was an increased expression of spinal cord specific Hox genes and decreased expression of brain related patterning transcription factor in spNPCs as compared to fbNPCs (FIG. 12G).

The Injured Spinal Cord Microenvironment has a Distinct Effect on the Proliferation and Differentiation of fbNPC and spNPCs

During CNS development, neurons, astrocytes and oligodendrocytes arise from common neuro epithelial progenitor cells in a process guided by the dynamic interplay of environmental signals(Silbereis et al., 2016). However, after SCI, these developmental cues are not present and different environmental factors are expressed.

This difference in the composition of differentiation factors between the injured and uninjured spinal cord microenvironment could have distinct effects on the proliferation and fate determination of NPCs that are from different developmental stages. To assess this effect, we exposed fbNPC and spNPCs in vitro to naïve spinal cord homogenate (Naïve-h) or injured homogenate (SCI-h). These homogenates were prepared from uninjured cervical spinal cord tissue or cervical injured tissue two weeks post injury.

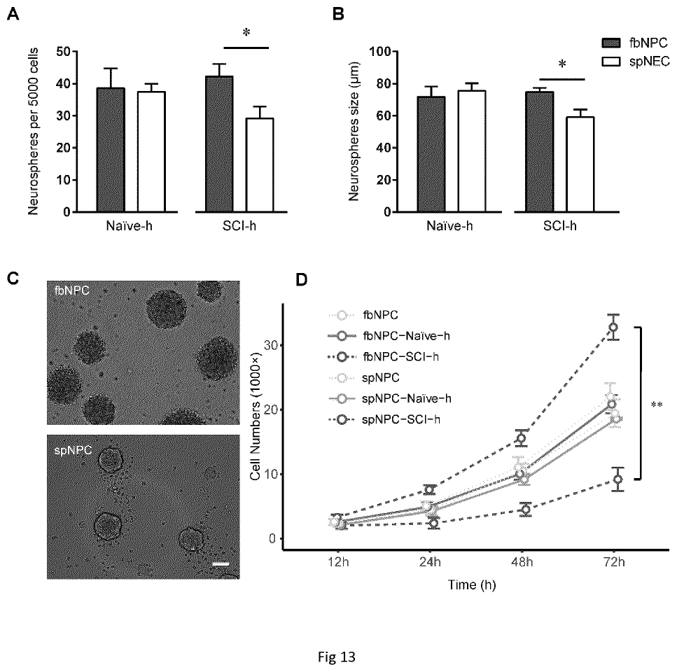

To investigate the effect of Naïve-h or SCI-h on the self-renewal ability of NPCs at different developmental stages, a clonal analysis was performed for fbNPC and spNPCs. Neurospheres were mechanically dissociated to a single cell suspension and plated at clonal density in the neurosphere assay (FIGS. 13A-C). There was no significant difference in the self-renewal ability of fbNPC and spNPCs when treated with Naïve-h, as the number of neurospheres generated from both lines was not significantly different (FIGS. 13A-C). Conversely, when treated with SCI-h, fbNPC generated significantly more neurospheres and demonstrated greater average neurosphere size as compared to spNPCs, suggesting that the proliferation rate of the fbNPC forming neurospheres was higher than spNPCs after exposure to SCI-h (FIGS. 13A-C). This was further confirmed using a BrdU assay (FIG. 13D).

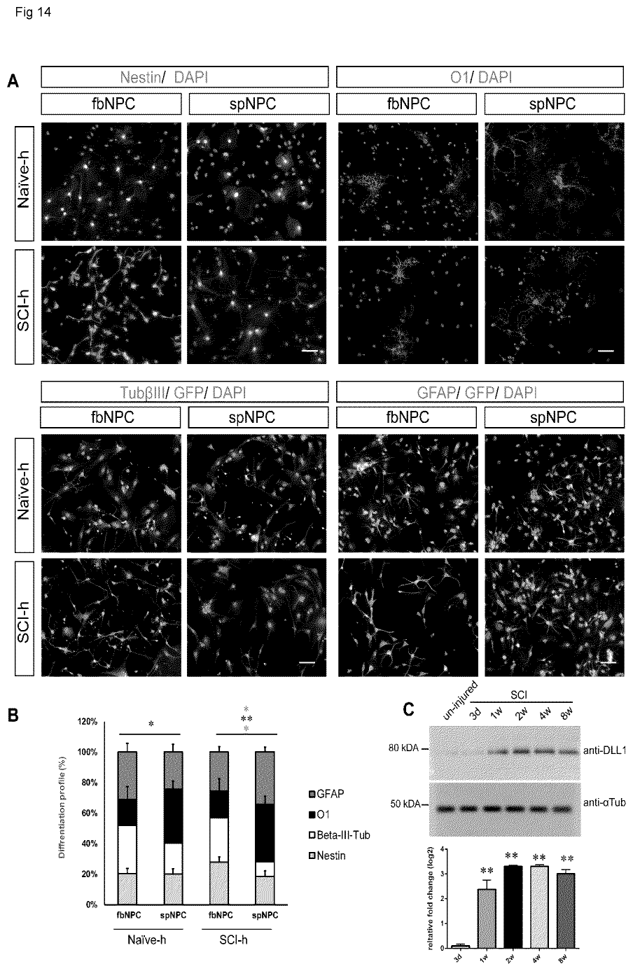

To explore the effect of factors present in the microenvironment of naïve or injured spinal cords on the differentiation of NPC lines, we differentiated cells in the presence of Naïve-h or SCI-h. Culturing NPC lines in the presence of Naïve-h for 4 weeks resulted in their differentiation to neurons (βIII-tubulin⁺), astrocytes (GFAP⁺) and oligodendrocytes (O1⁺), confirming their tripotency. In Naïve-h, fbNPC differentiated to more neurons (31.7±2.0%,) compared to spNPCs (20.5±1.9%, p<0.5), while spNPCs differentiated to more O1 expressing oligodendrocyte (fbNPC; 31.7±2.0% v.s. spNPCs; 20.5±1.9%, p<0.5). However, culturing in the presence of SCI-h had a distinct effect on fbNPC compared to spNPCs. A larger portion of fbNPC remained undifferentiated (Nestin⁺; 27.7±3.5%) or differentiated to neurons (29.4±2.8%), while spNPCs mainly differentiated along the astroglial and oligodendroglial lineage (O1⁺; 37.8±5.3, GFAP⁺34.0±3.1). Furthermore, a lower percentage of spNPCs remained undifferentiated (Nestin⁺; 18.29±3.8%) (FIG. 14).

Divergent Effects of Notch Signaling on fbNPC and spNPCs

Our results showed that SCI-h induces proliferation of NPCs when they are at an early developmental stage (fbNPC), while inducing their differentiation to an astroglial lineage when they are at a later developmental stage (spNPCs). This finding suggests that there are factors in the SCI niche which have different effects on early vs. late developmental stage NPCs.

Notch signaling is a pathway involved in binary cell fate decisions as well as induction or enhancement of terminal differentiation. During early CNS development, Notch signaling is first used to induce the self-renewal and proliferation of cells. However late in development, Notch induces the differentiation of cells to a glial fate(Grandbarbe et al., 2003; Namihira et al., 2009; Tanigaki et al., 2001). Therefore, Notch signaling is a potential candidate responsible for the observed developmental stage differences in differentiation after exposure to SCI-h. We analyzed the expression of Notch activating ligand, DLL1, in Naïve-h and SCI-h using western blotting and found that the expression of DLL1 was highly upregulated after SCI (FIG. 14C). DLL1 expression reaches its maximum 2 weeks after injury and begins to decrease around 2 months post injury. The distinct effect of Notch on developmentally different neural progenitors is correlated with their level of Pax6 expression(Sansom et al., 2009). Pax6 expression levels are higher in fbNPC compared to spNPCs.

The Pax6-regulated activity in neural stem cells is highly dose sensitive, with increasing Pax6 levels driving the system towards neurogenesis. Relative levels of Pax6 and Notch signaling are factors in a dynamic balance that controls whether neural stem cells self-renew, differentiate to neurons, or generate glial progenitor cells. In the presence of Notch signaling, increased Pax6 activity suppresses neurogenesis and promotes self-renewal, in part by repression of Neurog2. Conversely, decreased levels of Pax6 in the presence of Notch signaling result in gliogenesis.

fbNPC and spNPCs Survived, Migrated and Differentiated in the Injured Spinal Cord

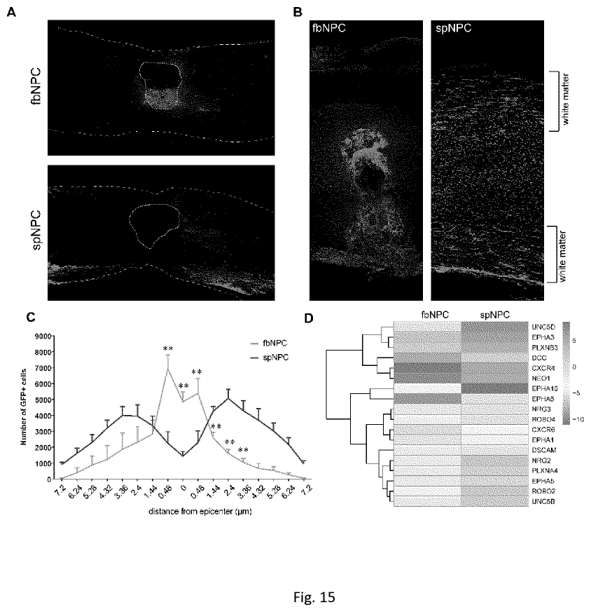

To investigate the in vivo behavior of fbNPC and spNPCs after transplantation, T-cell deficient RNU rats received a C6/7 cervical SCI followed by cell transplantation at 2 weeks post-injury. Transplanted cells (GFP⁺) were found in both the white and gray matter. Grafted fbNPC were located mainly around the lesion epicenter (FIGS. 15A,B) and showed a tendency to migrate towards the injury epicenter and to fill the cavity (FIGS. 15A,B). Conversely, spNPCs migrated as far as 7 mm rostral and caudal from the epicenter, and predominantly migrated along white matter tracts (FIGS. 15 A, C).

The chemotactic response of neural stem cells to the injury site is highly related to their differentiation status and the type of lesion(Filippo et al., 2013; Imitola et al., 2004). Chemokines, such as CXCL12, are expressed within the SCI epicenter and attract neural progenitors that express CXCL12 receptors, CXCR4 or CXCR7(Chen et al., 2015; Imitola et al., 2004). fbNPC express higher levels of chemoattractant receptors CXCR4 and CXCR7, cell adhesion molecules (e.g. CD44) and integrins (e.g. ITGA4), as compared to spNPCs, which may explain this chemotactic response (FIG. 15D)(Filippo et al., 2013; Imitola et al., 2004).

Quantification of GFP+ cells revealed extensive survival of transplanted cells within the injured spinal cord (FIG. 15E). The cell survival rate was higher in the spNPC group (11.2±4.6%) as compared to the fbNPC group (6.1±1.1%), although there was no statistically significant difference (FIG. 15E).

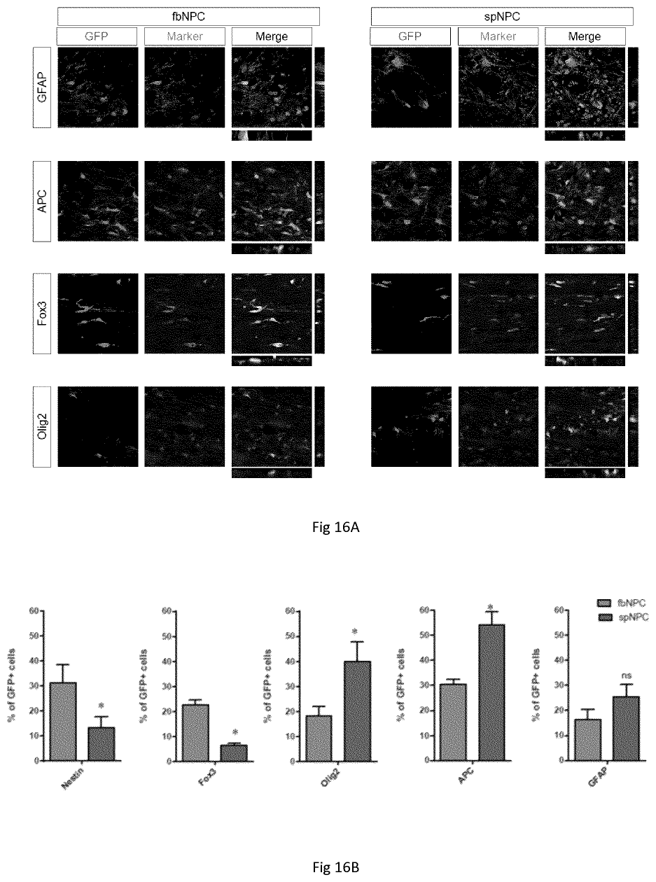

In terms of differentiation, both fbNPC and spNPCs differentiated into neurons, astrocytes, and oligodendrocytes in vivo, however, a proportion of cells in both lines remained in an undifferentiated Nestin positive state. The number of nestin positive cells for the fbNPC line was more than two times greater than spNPCs (fbNPC : 31.2±7.1 and spNPCs : 13.21±4.5%) (FIGS. 16A,B) in keeping with observed in vitro results after SCI-h exposure. The percentage of APC positive mature oligodendrocytes was significantly higher in transplanted spNPCs (54.23±5.24%) as compared with fbNPC (30.4±2.14%; p<0.05). Similarly, more immature Olig2 positive oligodendrocytes were observed in the spNPC group (spNPCs: 39.9±7.9 and fbNPC: 18.3±3.7%). The presence of GFAP positive astrocytes was not significantly different among transplanted spNPCs (25.4±5.1%) as compared to fbNPC (16.2±4.2%). In contrast, Fox3 positive neurons were significantly more abundant in transplanted fbNPC (22.6±1.9%) than spNPCs (6.5±0.8%) (FIGS. 16A,B).

We investigated the expression of Ki67 in GFP+grafted cells to identify

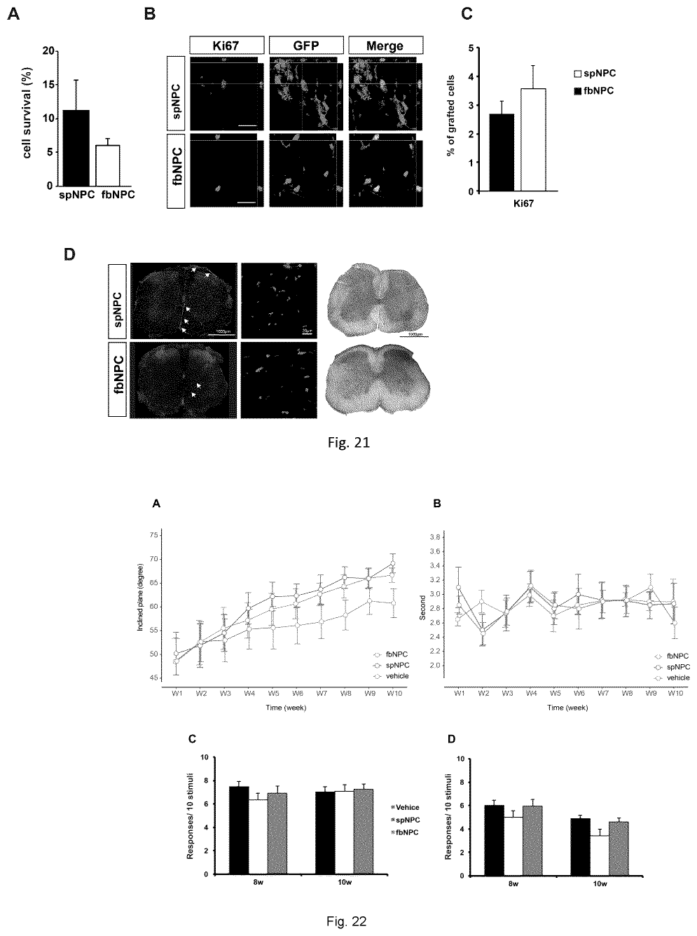

hyperproliferative or immature grafted cells (FIGS. 21A, B and C). The Ki67 positive rate was 2.67±0.48% and 3.56 ±0.80% in spNPCs and fbNPC, respectively, which was not significantly different (FIG. 21). This shows that even with a large proportion of undifferentiated Nestin+ cells, the graft is not hyperproliferative and the risk of tumorigenicity is low for both lines.

### Contribution of Transplanted Cells to Remyelination

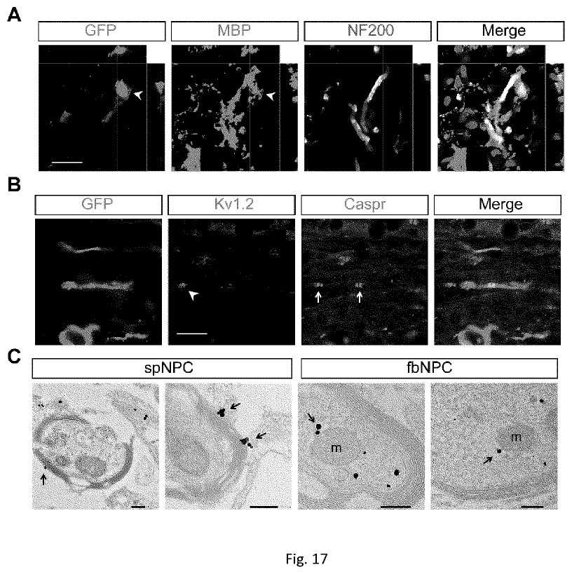

In order to be fully functional, the neurons derived from transplanted cells need to be myelinated. In addition to myelination, graft derived oligodendrocytes can contribute to the remyelination of denuded axons. To explore this, we performed immunohistological analyses using myelin basic protein (MBP) as well as NF200. Few GFP⁺ /MBP⁺ cells were observed in the fbNPC group, while numerous GFP⁺/MBP⁺ double-positive cells were observed in the spNPC group, suggesting that transplanted spNPCs differentiated into myelinating oligodendrocytes (FIG. 17A). Immunohistochemical analyses revealed that these GFP⁺/MBP⁺cells surrounded host neurofilament 200 (NF200⁺) axons (FIG. 17A). Furthermore, graft-derived myelination promoted the creation of nodes of Ranvier with expression of paranodal Caspr and the juxtaparanodal voltage-gated potassium channel Kv1.2 (FIG. 17B). We further evaluated myelination using Immunoelectron microscopy (FIG. 17C). Transmission electron microscopy showed that neurons derived from fbNPC were myelinated by endogenous myelinating cells. Conversely, in the spNPC group, immunogold-labelled GFP+ myelin lamellae were found to ensheath the spared rat axons (FIG. 17C). These results clearly demonstrated that grafted human spNPC-derived oligodendrocytes myelinate endogenous axons.

fbNPC and spNPC Transplantation Enhances Tissue Preservation After SCI

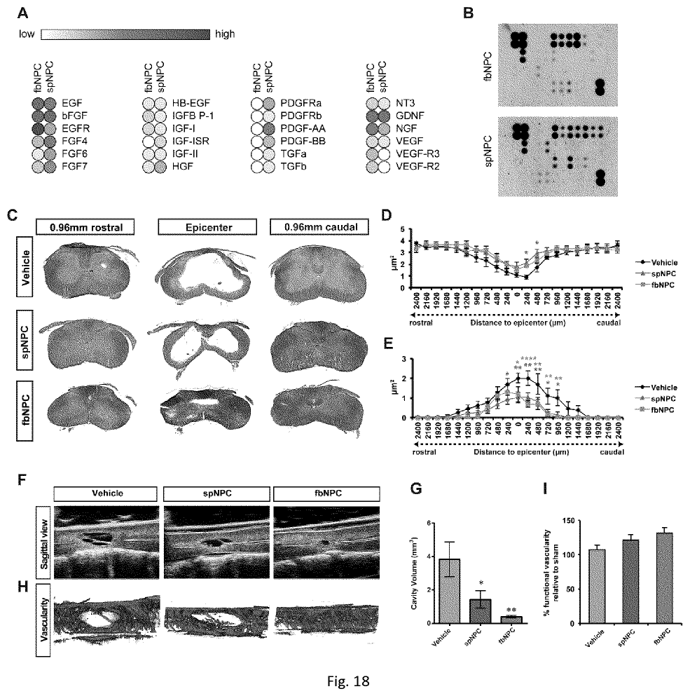

One of the main beneficial effects of transplanted NPCs is the secretion of trophic factors. Theses trophic factors have pro-neurogenic, pro-axonogenic, as well as various anti-apoptotic and pro-angiogenic effects, which preserve endogenous tissue(Ruff et al., 2012). To address the trophic factors which are secreted by fbNPC and spNPCs, we used a human growth factor antibody array to analyze the secretion of different growth factors between fbNPC and spNPCs. Both lines showed expression of different fibroblast growth factor (FGF) isoforms at an increased level. FGF isoforms have been linked to survival and neurite outgrowth in certain neuronal subtypes(Pataky et al., 2000). Also, pro-neurogenic trophic factors, such as NT3, NGF, and GDNF, were expressed by both lines at a high level. Interestingly, trophic factors that induce differentiation and proliferation of glial lineages, like PDGF and TGF isoforms, were increasingly expressed by spNPCs, while pro-angiogenic factors like VEGF were greatly expressed by fbNPC (FIGS. 18A,B).

Secretion of these trophic factors in vivo can enhance endogenous tissue preservation. Our histomorphometric analyses using LFB and H/E staining demonstrated that more white matter was present in spNPC treated animals as compared to animals treated with vehicle, but only at 240 and 480 μm caudal to the epicenter (FIGS. 18C,D). However the lesion was smaller in both cell transplantation groups (FIGS. 18C,E). The lesional tissue volume of spNPCs and fbNPC groups was significantly smaller compared with vehicle (vehicle: 3.82±0.45 mm3, spNPCs: 1.74±0.20 mm3, fbNPC: 1.89±0.26 mm3, p<0.01) (FIG. 18E).

In addition to this histomorphometric analysis, we further investigated cavitation and functional vascularity by using high resolution ultrasound (VHRUS) imaging(Soubeyrand et al., 2014). VHRUS imaging is capable of generating planar full-depth images and 3D reconstruction volumes of cavitation in situ. This helps to overcome difficulties with tissue shrinkage that occur during routine histological processing(Soubeyrand et al., 2014). The cavity size under VHRUS was significantly smaller in fbNPC and spNPC transplanted animals compared with vehicle treated animals (vehicle: 2.79±2.55 mm3, spNPCs: 1.37±0.25 mm3; p<0.05, fbNPC: 0.15±0.05 mm3; p<0.005, n=5, FIGS. 18F,G). The smaller cavity size in the fbNPC group arose not only from secretion of trophic factors by NPCs, but also from migration of fbNPC towards and filling the cavity. Interestingly, the vascularity was improved in the fbNPC group compared to vehicle (FIGS. 18H,I). Although this improvement was not significant, it reflects the higher secretion of pro-angiogenic growth factors by fbNPC.

spNPC-Derived Neurons Make Synaptic Connections with Endogenous Cells and Enhance Electric Conduction

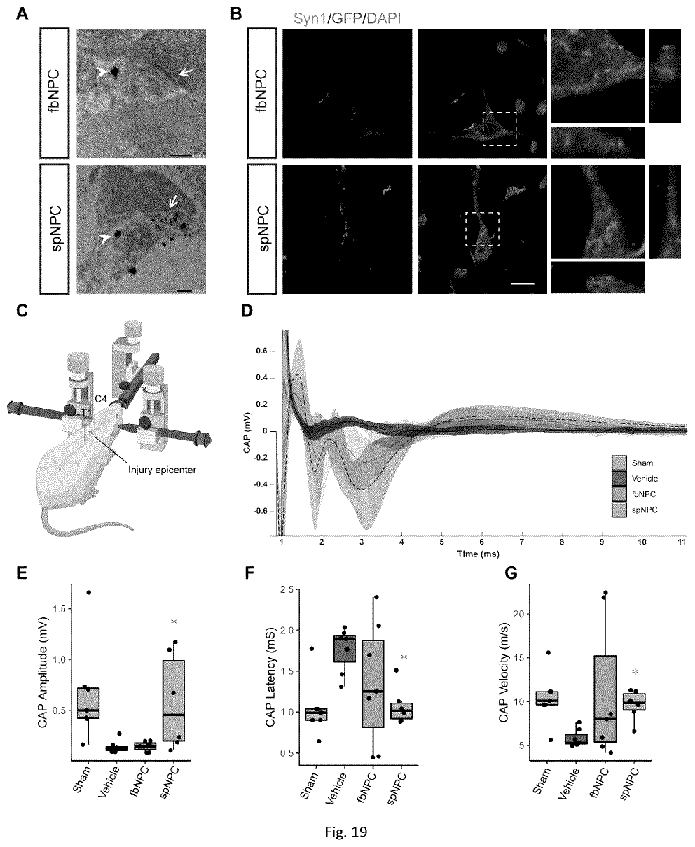

The neurons that are differentiated from transplanted cells must form synaptic connections with endogenous cells and integrate into local networks to promote functional recovery. Using immunostaining with Synapsin I (SYN1) antibody and immunotransmission electron microscopy, we assessed whether gold-labeled GFP+ cells formed synaptic connections with label-negative endogenous cells (FIG. 19). Both fbNPCs and spNPC were able to form synaptic connections. Formation of synaptic connections between fbNPCs or spNPCs and endogenous cells can contribute to functional recovery if they are electrically functional synapses. Specifically, these new connections could potentially contribute to greater electrical transmission across the injury site. To test this, we analyzed electrically evoked compound action potential (CAP) transmission across the injury site (C5 to T1). The CAP amplitude was significantly higher in the spNPC transplant group compared to the control fbNPC group (FIG. 19C). CAP measurements of the dCST across the spinal cord lesion showed that animals receiving spinal NPCs had higher CAP amplitudes on average (0.6±0.5 mV), compared with animals receiving fbNPCs (0.1±0.05 mV) or the vehicle group (0.1±0.06 mV). On average, higher dCST conduction velocities were observed for the animals that received NPCs (9.6±1.7 ms for spNPCs and 10.8±7.9 ms for fbNPCs) compared with the vehicle group (5.8±1.0 ms) (FIG. 19D-G).

Transplantation of fbNPC and spNPCs Improves Functional Recovery

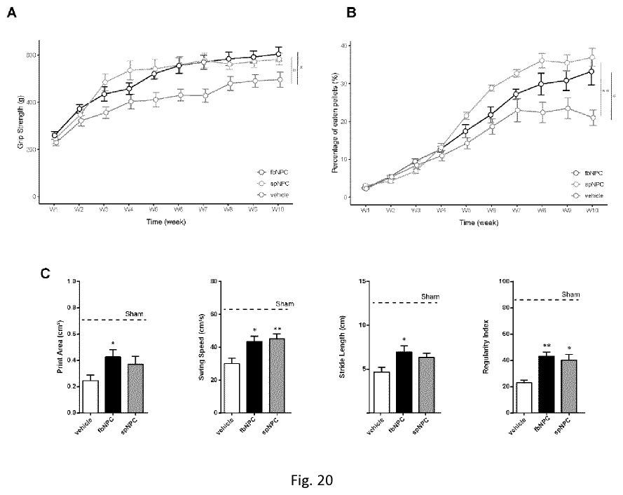

Next, we investigated whether transplantation of fbNPC and spNPCs was associated with functional recovery. Forelimb strength and trunk stability were assessed with grip strength and inclined plane behavioral tasks(Wilcox et al., 2017). All injured animals consistently recovered forelimb grip strength over the assessment period, although recovery trajectories diverged at approximately 4 weeks post-transplantation. There was a significant improvement in forelimb grip strength in both fbNPC and spNPC groups compared to the vehicle control group (p<0.05) (FIG. 20A). Although we measured a trend toward better recovery in both groups for the inclined plane test, the recovery was not significantly different as compared to vehicle (FIG. 20B). Using the CatWalk (Noldus Inc.) digital gait analysis system, we also quantified several static and dynamic parameters of locomotion relevant to cervical SCI at 8 weeks post transplantation. All injured groups exhibited abnormal walking patterns, slow rates of locomotion, and abnormal paw prints (FIG. 19C). Forelimb swing speed was significantly improved in rats transplanted with both fbNPC and spNPCs versus the vehicle control (FIG. 20C). While the forelimb print area and stride length were not significantly improved in spNPC transplanted animals, they improved significantly for the fbNPC group. Furthermore, the regularity index, which is an indicator of coordination between all four limbs, was significantly improved for both cells lines (FIG. 20C).

Increased neuropathic pain after cell-based treatment is a potential concern(Hofstetter et al., 2005). Hence, we assessed thermal and mechanical allodynia (experimental procedure in supplemental information). Latency times for the rat to remove its tail from a focal heat source (FIG. 22A) were not significantly different at any time point among the groups. In addition, there was no significant difference in the responses to von Frey filament application to the plantar surface of fore and hind paws at 8 and 10 weeks after injury (FIGS. 22B,C). These data indicate that the cell-transplantation therapy does not increase neuropathic pain in our model.

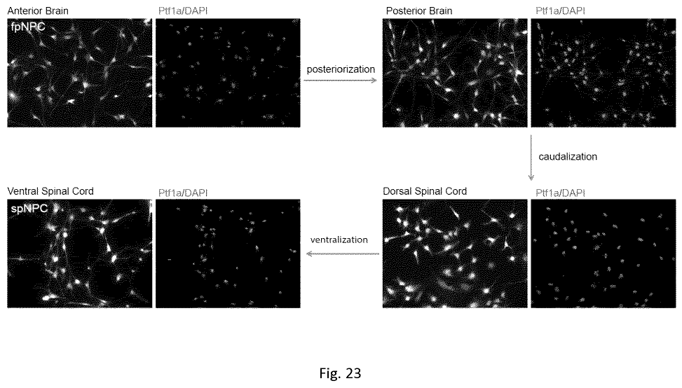

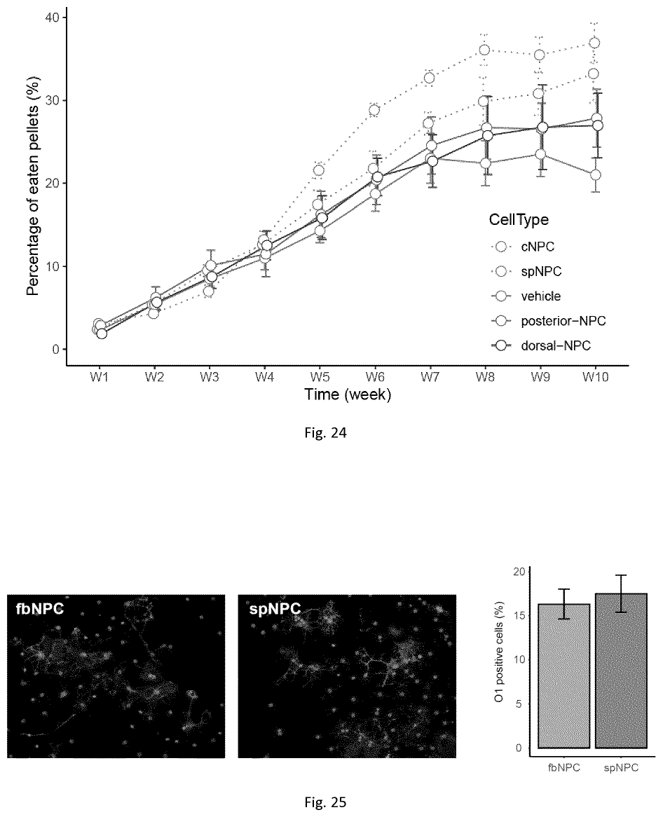

We have chosen to compare cells with anterior brain vs. ventral spinal cord identity because these cells demonstrated lower expression of Ptf1a (FIG. 23) compared to their posterior/dorsal counterparts. Ptf1a is a transcription factor that induces differentiation to GABAergic inhibitory neurons in the posterior brain (cerebellum) and spinal cord dorsal horn. We have demonstrated no significant involvement in functional recovery compared to vehicle (FIG. 24). One potential reason for this finding could be higher differentiation to GABAergic neurons. Although GABAergic neurons have been shown to be important for balancing neural networks and the modulation of neuropathic pain, transplanting cells with the potential to primarily differentiate to inhibitory neurons may not be effective for relaying the signal.

We demonstrated that the expression of BMPs, TFGβ, Jagged1/2 and Notch protein are upregulated in our model of RNU rat cervical SCI 2 weeks after injury relative to the uninjured (naïve) cervical spinal cord. When we culture the cells in baseline differentiation medium, the number of cells differentiating to O1+ cells was not significantly different for spNPCs as compared to fbNPCs (FIG. 25).

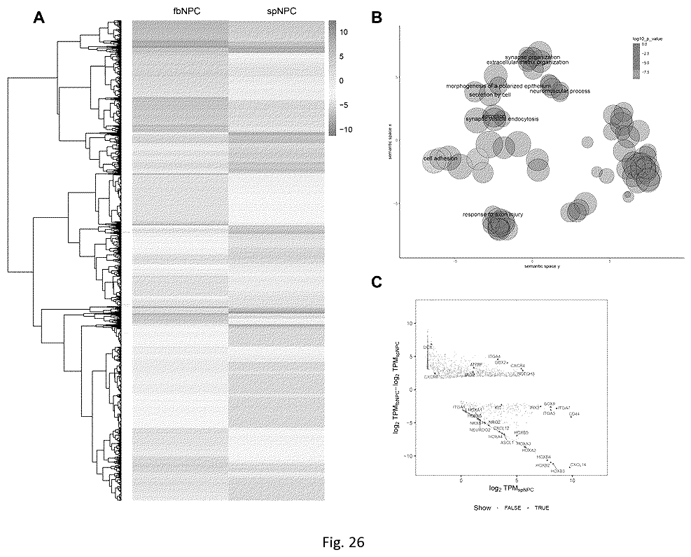

However, to have a global understanding of the gene expression profile for fbNPCs and spNPCs, we performed RNAseq analysis between the two lines. We demonstrated that fbNPCs express higher levels of chemoattractant receptors CXCR4 and CXCR7, cell adhesion molecules (e.g. CD44) and integrins (e.g. ITGA4), as compared to spNPCs, which may explain this chemotactic response. (FIG. 26).

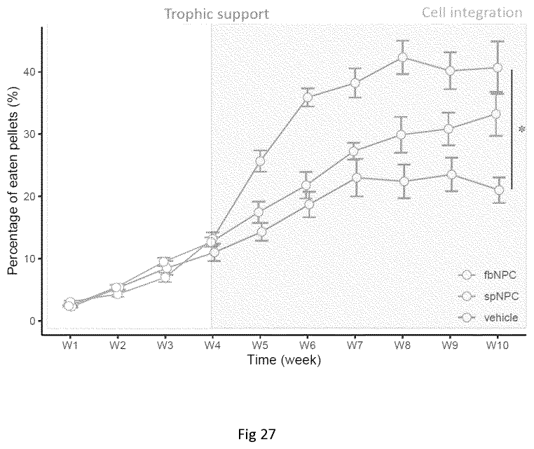

Understanding various mechanisms involved in the obtained functional recovery is important and has implications for the design of regenerative therapies for SCI. In general, trophic support has a faster effect on functional recovery, therefore the functional recovery that is emerged in the first 2-3 weeks after transplantation, is most probably attributed to trophic support. It takes more than 3 weeks for human cells to differentiate to functionally active neurons or myelinating cells7. Therefore, the portion of the recovery that is emerged after this time, can have an additive effect of both trophic support and cell integration (FIG. 27).

The optimum temporospatial developmental stage of neural stem/progenitor cells must be determined in order to produce cell transplantation strategies with the highest likelihood of improving outcomes post-SCI. In the current study, we compared the therapeutic potential of hiPSC-derived neuroepithelial stem progenitor cells at two distinct stages of their development in a cervical model of SCI. fbNPC represent a very early stage of development of neural progenitor cells, which have anterior identity, while spNPCs demonstrate a later stage of development of neural progenitors with a caudal ventral identity. Our results demonstrated that both cell types contributed to functional recovery, but through different mechanisms.

Understanding the reciprocal interaction between the SCI microenvironment and cells with different identities will allow the field to more closely target the graft to a patient's requirements. The injured spinal cord microenvironment showed distinct effects on the proliferation and differentiation of fbNPC and spNPCs. The self-renewal potential of fbNPC increased upon exposure to the SCI niche, while this environment biased the differentiation of spNPCs towards a glial linage. This contradictory effect is a typical hallmark of Notch signaling on primitive vs. definitive neural progenitors (Grandbarbe et al., 2003; Namihira et al., 2009; Salewski et al., 2013; Tanigaki et al., 2001). This is in concordance with activation of Notch signaling in the SCI microenvironment (FIG. 14C). The expression level of Pax6 is higher in fbNPC compared to spNPCs (FIG. 12D). The dynamic balance for Pax6 expression and Notch signaling specifies self-renewal over differentiation to glial progenitors, or vice versa (Sansom et al., 2009).

Here, we demonstrate that the interplay between graft and host tissue is two-sided. Transplanted NPCs produced effects on the injured tissue through both cell-autonomous (cell replacement) and noncell-autonomous (trophic) mechanisms. Both NPC lines differentiated to neurons, oligodendrocytes and astrocytes, although in different proportions. Differentiated fbNPC mainly consisted of neurons, while spNPCs predominantly differentiated to oligodendrocytes. Importantly, the NPC derived neurons could potentially form synapses with endogenous neurons and improve neural circuit conduction(Lu et al., 2012, 2014) by bridging and relaying supraspinal neurons from the cortex to their spinal targets(Bonner and Steward, 2015).

The NPC derived oligodendrocytes could potentially contribute to remyelination. spNPCs mainly differentiated to Olig2 and/or APC positive oligodendrocytes. These cells could differentiate to MBP positive myelinating oligodendrocytes (FIG. 17B), and our EM analysis showed that oligodendrocytes derived from transplanted spNPCs could contribute to the remyelination of denuded endogenous axons (FIG. 17B). After SCI, varying degrees of demyelination occur (Karimi-Abdolrezaee et al., 2006; Nashmi and Fehlings, 2001) and graft derived remyelination has an important impact on functional recovery(Karimi-Abdolrezaee et al., 2006; Salewski et al., 2015). Although some degree of spontaneous remyelination by endogenous oligodendrocyte precursor cells or migrating Schwann cells can occur (Franklin and Hinks, 1999; Stange) and Hartung, 2002), the extent is limited due to the minimal proliferation of myelinating cells (Li et al., 1996).

After SCI, cystic cavities form as a result of cell death and the clearance of damaged tissue by phagocytotic cells(Dusart and Schwab, 1994; Liu et al., 1997). This damage to the spinal cord significantly affects functional recovery. Other than replacing lost cells, transplanted NPCs (mainly fbNPC) contributed to forming a cellular bridge through the cystic cavity. This cellular bridge provides a structural substratum that allows injured axons to grow into a permissive environment.

Migration towards the cavity (pathotropism) is very important for forming this cellular bridge. However, the chemotactic responses of neural stem cells to the injury site is related to their differentiation status and expression of receptors for chemoattractants (Filippo et al., 2013; Imitola et al., 2004). Chemoattractants, like CXCL12 (SDF1), are expressed by the injured site post-SCI, and attract cells that express CXCL12 receptors, CXCR4 or CXCR7(Chen et al., 2015; Imitola et al., 2004). It has been shown that neural stem/progenitor cells at their early stages of development show higher expression of these receptors and a higher degree of pathotropism (Chang et al., 2013; Ferrari et al., 2012). This is in concordance with our results showing that early stage NPCs (fbNPC) express higher levels of CXCR4 and CXCR7 than spNCEs, and demonstrate a higher tendency to migrate towards the cavity and form a cellular bridge.

In addition to cell replacement, transplanted NPCs provide trophic support to the injured tissue. Secretion of trophic factors promotes tissue repair by preventing tissue damage and enhancing the survival of endogenous neural cells. It also re-establishes the functional interactions between neural and glial cells(Ruff et al., 2012). Both NPC lines secreted elevated levels of trophic factors like FGF, NT3, NGF, and GDNF, which are involved in the survival and regeneration of injured tissue. The composition of secreted growth factors was slightly different between the cell types, with spNPCs expressing more pro-oligodendrogenic factors, which helps remyelination, while fbNPC expressed more pro-angiogenic factors that help restore vascularity in the injured tissue.

As research continues into strategies to generate neural stem/progenitor cells with different identities, recognizing the optimal differentiation state of cells for transplantation to treat SCI is vital for clinical translation. Overall, our study demonstrates that transplantation of human fbNPC and spNPCs can exert positive effects through trophic support and cell replacement. Both fbNPC and spNPCs secrete a plethora of different trophic factors. A proportion of the functional recovery observed in fbNPC transplanted animals could be attributable to providing a permissive cellular bridge in the cavity as well as integration and connection of fbNPC derived neurons into the host neural network. Conversely, a large proportion of spNPCs' effects appear to be the result of enhanced remyelination. Both mechanisms are important for functional recovery. Further experiments are required to determine the synergistic effect of transplanting a combination of both fbNPC and spNPCs.

### Experimental Procedures

Generation and Characterization of fbNPC and spNPCs

Human iPSCs (hiPSCs) line PB1-53, as described before(Hussein et al., 2011), were differentiated to NPCs using dual SMAD inhibition in monolayer culture with some modifications(Varga et al.). fbNPC were maintained in DMEM:F12 supplemented with N2, B27-VA media with FGF2 (10 ng/ml) and a combination of inhibitors targeting TGFβ (SB431542) and WNT signaling (CHIR99021)(Payne et al., 2018; Varga et al.). For spNPCs, we patterned cells with stepwise treatment with patterning factors (Lippmann et al., 2015). At the first step cells were cultured in DMEM:F12 media supplemented with B27, N2, FGF2 (20 ng/ml), FGF8 (200 ng/ml) for three days(Lippmann et al., 2015). Cells were then caudalized by supplementing the culture with 0.1 μM retinoic acid agonist EC23 for an additional 5 days. Cells underwent ventralization by treatment with 1 μM sonic hedgehog (Shh) agonist Purmorphamine for 3 days. At this stage cells showed a ventral spinal cord identity. spNPCs were maintained in media consisting of B27, N2, FGF2 (10 ng/ml), EGF (10 ng/ml) and 740Y-P (1 μM) for three passages prior to transplantation. During passaging, 10 μM Rock inhibitor (Y-27632) was added on day 1. Further details are provided in Example 1.

mRNA Expression Profiling

Quantitative RT-PCR was used to examine the expression profile of fbNPC and spNPCs. mRNA was isolated using the RNeasy Mini Kit (Qiagen74104). A Nanodrop spectraphotometer was used to evaluate the concentration and purity of the mRNA. cDNA was synthesized using a SensiFAST™ cDNA Synthesis Kit (Bioline 65053) with random hexamere primers. RT-PCR was performed using TaqMan design primers with SensiFAST Probe Hi-ROX master mix (Bioline 82020) under recommended thermocycling parameters on a 7900HT Real time PCR system. Samples were run in triplicate. Values were normalized to the GAPDH housekeeping gene. For the examination of the gene expression levels, results were normalized to GAPDH and to the reference cells. The gene expression level was calculated using the 2-^{ΔΔCT} method.

### Preparation of Spinal Cord Homogenate

Two weeks after injury, rats were perfused with ice-cold PBS, and a 5 mm long segment of the injured spinal cord centered on the injury epicenter was removed. For naïve rats, the same region of the spinal cord as the injured rats was removed for this preparation. Spinal cords of 5 rats for each group were pooled together in 1 ml DM EM:F12. The tissue was homogenized by using a small conical mortar and pestle for 2 min, while kept on ice. The homogenate was cleared by centrifugation at 12000 g for 15 min, and the total protein concentration of cleared supernatants was measured using a BCA test. After adjusting the total protein concentration, the aliquots were kept at −80° C. until use.

In vitro Treatment with Spinal Cord Homogenate

In order to study the effect of injury induced factors on the differentiation potential of NPC lines in vitro, NPCs were cultured on their maintenance media without growth factors (no FGF2 for fbNPC and no FGF2/EGF for spNPC) and treated with 100 μg/ml cleared homogenate from the injured (SCI-h) or naive (Naive-h) spinal cord for 30 days. Cells were then fixed for 20 min with 4% paraformaldehyde in phosphate-buffered saline (PBS) and 40% sucrose at room temperature. Following fixation, cells were permeabilized in 0.1% Triton X-100 and 0.1% sodium citrate in PBS for min (except for O1 staining) and then placed in blocking buffer (5% BSA) for 1 h. Primary antibodies were diluted in a blocking buffer solution and incubated with the cells overnight at 4° C. Following extensive washing, samples were incubated with fluorophore-conjugated secondary antibodies for 1 h.

### Human Growth Factor Antibody Array

To explore the difference in trophic factors secreted by fbNPC and spNPCs, a membrane-based antibody array (Abcam, #ab134002) was used to compare the expression of 41 growth factors in conditioned medium prepared from cells. fbNPC and spNPCs were seeded at an equivalent density of 1×10⁶ cells on 10 cm laminin coated plates, and after 18 h the conditioned media were collected. The conditioned media was incubated with the antibody array and developed with biotin-conjugate antibodies and HRP-Streptavidin, as per the manufacturer's instructions.

### Neurosphere Assay

fbNPC and spNPCs were plated in their respective maintenance media w/wo Naïve-h or SCI-h (100 μg/ml) at a clonal density of 10 cells/μL in a final volume of 500 μl medium in uncoated 24-well plates (Nunc, Rochester, N.Y.). Neurospheres >50 μm in diameter were quantified after 7 days of undisturbed culture. Just prior to imaging, the content of each well was transferred to a Matrigel coated dish, incubated for 30 min and fixed with 4% PFA.

### Cell Proliferation Assay

fbNPC and spNPCs were plated on laminin coated 96-well tissue culture plates (removable strip plates, Corning) at a density of 1×10³ cells/100 μl/well and the cell number was determined at 12, 24, 48 and 72 hours after plating using a BrdU cell proliferation assay (Abcam, #ab126556), as recommended by the manufacturer.

### Animal Care

All animal experiments were approved by the Animal Care Committee, University Health Network (Toronto, Ontario, Canada) in accordance with the policies of the Canadian Council of Animal Care for the use of experimental animals and under the supervision of clinical veterinarians. Adult Rowett Nude (ATN) rats 180-230 g (Charles River Laboratories) were used for cell transplantation.

### Surgical Procedure to Induce Rat Spinal Cord Injury

The clip compression model of cervical SCI has been characterized extensively in our laboratory and described previously(Wilcox et al., 2014). Briefly, ATN rats were anesthetized using 4% isoflurane and administered 0.05 mg/kg buprenorphine and 10 ml saline, and were sedated for the remainder of the surgery under 2% isoflurane. The rats received a C6 and C7 laminectomy. A modified aneurysm clip calibrated at 21.5 g (Walsh, Oakville, Ontario, Canada) was applied extradurally to the spinal cord at the C6 vertebral level for 60 seconds and then removed. For the sham group, only a laminectomy was performed without clip compression. Gel foam (Ferrosan, Denmark) was placed on the spinal cord at the end of the surgical procedure and the incision was closed in layers using standard silk sutures. Animals were allowed to recover in their cage under a heat-lamp and, subsequently, were housed in a 12-hour light-dark cycle at 26° C. with free access to food and water. Animals received extensive postoperative care, including Clavamox in drinking water 3 days before injury until the study endpoint. Animals were administered buprenorphine (0.1 mg/kg) for 3 days and meloxicam (1.0 mg/kg) for 3 days. Injured rats were administered fluids and nutritional support, and their bladders were manually voided three times daily for 14 days as needed.

### Intraspinal Transplantation

Animals were randomly divided into three groups to receive transplantation of 4×10⁵ fbNPC, spNPCs or a vehicle (control) injection, on day 14 post-injury. Under isoflurane (1-2%) and a 1:1 mixture of O₂/N₂O, rats were placed in a stereotactic frame and administered 0.05 mg/kg buprenorphine and 10 ml saline, and injuries were carefully re-exposed. Cells were made ready for transplantation by treating the monolayer of both NPC lines with Accutase (0.05 ml/cm² cell culture area) and were incubated at 37° C. for 2 min. The Accutase was then neutralized by media, cells were detached and spun at 400 g for 4 mins, re-suspended in culture media and viable cells counted. Cells were transplanted intraspinally at a density of 50,000 cells/μl. 2 μl of cell suspension were injected per site and there were four injection sites per rat (1.0 mm bilateral to the midline at 2 mm rostral and caudal). Injections were delivered at 0.6 μl/minute, left to dwell for 2 minutes, and retracted over 2 additional minutes using a Hamilton syringe and a stereotaxic injection system (System UMP3 with Micro4, World Precision Instruments, Sarasota, Fla.). The control animals also received the same number of injections to the spinal cord with only culture media.

### Tissue Processing and Lesion Morphometry

At 10 weeks post-injury, rats were deeply anesthetized with isofluorane and transcardially perfused with 180 ml of ice-cold phosphate-buffered saline (PBS) and 180 ml of 4% paraformaldehyde in PBS. Following collection, spinal cord tissue was fixed for 5 hours and then cryoprotected in 30% sucrose in PBS for 24 hours. Spinal cord segments were embedded, frozen and stored at −80° C. Cryostat sections were made 30 μm thick along a 1.5 cm length portion of the spinal cord, and centered rostrocaudally at the site of injury.

Serial sections were stained with Luxol Fast Blue (LFB) and Hematoxylin & Eosin (H&E). LFB is a myelin-selective stain, while H&E are stains for all cell nuclei and cytoplasmic proteins, respectively(Nguyen et al., 2012). Slides were retrieved from −80° C., baked at 56° C. for 15 minutes and washed in various medium to prepare for overnight LFB staining at 56° C. The following day, tissue was retrieved from the oven and processed through various washing steps for H&E staining. Afterwards, the tissue was sequentially dehydrated with increasing concentrations of alcohol, xylene and cover slipped.

A blinded investigator performed LFB and H&E analyses on tissue±2,440 μm centered at the injury epicenter. Unbiased measurements were made using a Cavalieri volume probe from Stereo Investigator (MBF, Bioscience, Wilson, Vt.) to produce area and volume estimations of preserved white matter and lesional tissue(Wilcox et al., 2014). Lesional tissue was defined as areas with the following aberrant histology; small round cysts, irregularly shaped vacuoles, disorganization of both white and gray matter and eosinophilic neurons. Calculations and analyses were done for tissue sections every 240 μm.

In vivo Very High Resolution Ultrasound (VHRUS) Imaging

VHRUS and Power Doppler imaging were performed as previously described(Soubeyrand et al., 2014). Under isoflurane anesthesia, animals were placed within a custom-made stabilization frame on the imaging platform (Vevo imaging station, Visualsonics, Toronto, Canada). The injury was re-exposed through a mid-line incision and ultrasound gel (Scanning Gel, Medi-Inn, Canada) was placed on the dura mater. The spinal cord was scanned with the VHRUS probe (44 MHz, Vevo 770, Visualsonics, Toronto, Canada). The 3D B-mode scans were analyzed using ImageJ software and the TrakEM2 plugin to generate a reproducible cavity volume.

For Power Doppler analysis, a field-of-view with fixed area was defined and centered manually on the central sagittal slice. The Doppler signal was binarized through image thresholding, and batch analysis was carried out for all sagittal sections. The area fraction of positive Doppler signal was multiplied to the actual image area to yield a Doppler-positive area per sagittal slice, which was ultimately summed to yield the total Doppler area for each spinal cord (termed “functional vascularity”). For clarity, all values have been normalized to the sham injury Doppler signal.

### Immunohistochemistry and Quantification

Immunohistochemistry was performed as previously described(Wilcox et al., 2014). The cryostat sections were first incubated in PBS for 5 min, prior to 1 h incubation in blocking buffer (1% BSA, 5% Skim milk, and 0.3% Triton-X 100 in PBS) in RT. After removing the blocking solution, primary antibodies were incubated overnight at 4° C., followed by washing three times with PBS for 10 min (Table S1). Slides were rinsed and incubated in appropriate fluorescent secondary antibodies (all 1:500, Thermo Fisher Scientific) for 2 h at room temperature. The images were taken using a Zeiss LSM 510 or LSM880 confocal microscopes.

### Immuno-Electron Microscopy

Frozen sections were incubated with anti-GFP mouse monoclonal antibody, and then incubated with nanogold-conjugated anti-mouse IgG secondary antibody (1:100 Invitrogen). Sections were fixed with 2.5% Glutaraldehyde, post-fixed with 0.5% OsO4 and embedded into Epon. Ultrathin sections (70 nm thick) were prepared, stained with uranyl acetate and lead citrate, and observed under a transmission electron microscope (TEM, JEOL 1400 plus). For the analysis of the synaptic density and the ratio of asymmetrical/symmetrical synapses, sets of micrographs for 4 rats in the control-hiPSC-NPC group and 4 rats in the GDNF-hiPSC-NPC group were used. The synaptic counts were expressed as the number of synapses on a membrane length unit of 100 μm. The estimate of synaptic parameters was derived from 145 synapses in hiPSC-NPC and 185 synapses in the GDNF-hiPSC-NPC group. By using well-established criteria for symmetric and asymmetric synapses (Gray, 1959), we counted the number of each type of synapse on electron microscopy images.

Recording in vivo Compound Action Potentials

Electrophysiological recordings were carried out under isoflurane anesthesia (1.0-1.5% inspiratory concentration). Twisted bipolar stimulation electrodes were made with polyimide-insulated stainless-steel wires with outer diameter of 0.2 mm and electrode-tip spacing of 0.1 mm (Plastics One, Roanoke, Va., USA). An aCSF-filled glass microelectrode with a 100˜120 μm tip diameter was used for recordings. Stimulation was applied at spinal cord segment T1 at 1.2 mm depth, targeting the dorsal corticospinal tract (dCST). Stimulation protocol consisted of delivering a cathodic rectangular wave with a pulse width of 0.1 ms and an amplitude of 1.5 mA, every 10 seconds. Stimulation pulses were generated using a PSIU6 stimulus isolation unit Grass S88 stimulator (Grass Technologies, USA). Evoked compound action potentials (CAPs) were recorded from spinal cord segment C4 at 1.2 mm depth, also targeting the dCST (Li et al., 2016). The spacing between the stimulating and recording electrodes was 10 mm. CAPs were recorded in DC mode with Axoprobe 1A amplifier (Molecular Devices, CA, USA) and processed using pClamp8 software and Digidata 1320A (Molecular Devices, CA, USA) with the sampling rate of 83.33 kHz. 2 kHz low-pass and 100

Hz high pass filters were used for these recordings. The data analysis was performed offline using a custom written program in Matlab (Math Works, Natick, Mass., USA) to measure the peak-amplitude and peak-latency of the evoked CAP responses in each animal. CAP conduction velocity was calculated by dividing the electrodes' spacing (10 mm) by the measured peak-latency.

### Behavioral Assessment

Rats from each experimental group underwent weekly neurobehavioural testing to assess forelimb strength, digital dexterity, trunk stability and function. Two independent, blinded observers measured each parameter. Forelimb strength was assessed using a force meter to measure grip strength(Garcia-Alias et al., 2009). Trunk and forelimb strength were also assessed using the inclined plane test(Bresnahan et al., 1987), which required rats to hold their standing position while the angle of the surface beneath them was incrementally increased. The test finished when the rats were no longer able to hold their position for 5 seconds. The last angle that rats were able to hold for 5 seconds was recorded. Forelimb gait analysis was performed using the Catwalk gait assessment system (Noldus Information Technology, Wageningen, Netherlands)(Dai et al., 2011),(Miyagi et al., 2011).

### Statistical Analyses

Results are stated as mean±standard error of the mean (SEM) or Standard deviation (SD) as indicated in the figure legends. Neurosphere assay and immunohistological data were analyzed using Student's t-tests. Histomorphometric and behavioral data were analyzed using two-way analysis of variance (ANOVA) with Tukey's post hoc test, or one-way ANOVA with Turkey's post hoc test as indicated in figure legends. The significance level of all analyses was set at p<0.05. Data were analyzed with Prism 6 (GraphPad Software, San Diego, Calif.).

### Example 3

Spinal -NPCs will be dissociated into a single-cell suspension at a concentration ranging from 1×10 \hat{ }4 to 1×10\hat{ }6 cells/μl in any vehicle (can be saline or any FDA approved vehicle) or cell can be encapsulated in a biomaterial/scaffold or matrix to fill in the lesion cavity. Cells will be stereotactically injected into spinal cord in defined rate

### REFERENCES

1. Tetzlaff, W. et al. A systematic review of cellular transplantation therapies for spinal cord injury. J. Neurotrauma 28, 1611-1682 (2011).
2. Ryu, J. K., Cho, T., Wang, Y. & McLarnon, J. G. Neural progenitor cells attenuate inflammatory reactivity and neuronal loss in an animal model of inflamed AD brain. J. Neuroinflammation 6, 39 (2009).
3. Ziavra, D. et al. Neural stem cells transplanted in a mouse model of Parkinson's disease differentiate to neuronal phenotypes and reduce rotational deficit. CNS Neurol. Disord. Drug Targets 11, 829-835 (2012).
4. Cohen, M. E., Fainstein, N., Lavon, I. & Ben-Hur, T. Signaling through three chemokine receptors triggers the migration of transplanted neural precursor cells in a model of multiple sclerosis. Stem Cell Res. 13, 227-239 (2014).
5. Bacigaluppi, M. et al. Neural Stem Cell Transplantation Induces Stroke Recovery by Upregulating Glutamate Transporter GLT-1 in Astrocytes. J. Neurosci. Off. J. Soc. Neurosci. 36, 10529-10544 (2016).
6. Lepore, A. C. & Maragakis, N. J. Targeted stem cell transplantation strategies in ALS. Neurochem. Int. 50, 966-975 (2007).
7. Pluchino, S. & Martino, G. The therapeutic plasticity of neural stem/precursor cells in multiple sclerosis. J. Neurol. Sci. 265, 105-110 (2008).
8. Madrazo, I. et al. Transplantation of Human Neural Progenitor Cells (NPC) into Putamina of Parkinsonian Patients: A Case Series Study, Safety and Efficacy Four Years after Surgery. Cell Transplant. 28, 269-285 (2019).
9. Levine, A. J. & Brivanlou, A. H. Proposal of a model of mammalian neural induction. Dev. Biol. 308, 247-256 (2007).
10. Kim, J. Y. et al. Identification of molecular markers distinguishing adult neural stem cells in the subventricular and subcallosal zones. Anim. Cells Syst. 21, 152-159 (2017).
11. Hodge, R. D. & Hevner, R. F. Expression and actions of transcription factors in adult hippocampal neurogenesis. Dev. Neurobiol. 71, 680-689 (2011).
12. Tian, C. et al. Foxg1 has an essential role in postnatal development of the dentate gyrus. J. Neurosci. Off. J. Soc. Neurosci. 32, 2931-2949 (2012).
13. Ghazale, H. et al. RNA Profiling of the Human and Mouse Spinal Cord Stem Cell Niches Reveals an Embryonic-like Regionalization with MSX1+ Roof-Plate-Derived Cells. Stem Cell Rep. 12, 1159-1177 (2019).
14. Chambers, S. M. et al. Highly efficient neural conversion of human ES and iPS cells by dual inhibition of SMAD signaling. Nat. Biotechnol. 27, 275-280 (2009).
15. Kumamaru, H. et al. Generation and post-injury integration of human spinal cord neural stem cells. Nat Methods 15, 723-731 (2018).
16. Kadoya, K. et al. Spinal cord reconstitution with homologous neural grafts enables robust corticospinal regeneration. Nat. Med. (2016) doi:10.1038/nm.4066.
17. Mothe, A. J., Zahir, T., Santaguida, C., Cook, D. & Tator, C. H. Neural stem/progenitor cells from the adult human spinal cord are multipotent and self-renewing and differentiate after transplantation. PloS One 6, e27079 (2011).
18. Yuan, F. et al. Efficient generation of region-specific forebrain neurons from human pluripotent stem cells under highly defined condition. Sci. Rep. 5, 18550 (2016).
19. Su, Z. et al. Antagonism between the transcription factors NANOG and OTX2 specifies rostral or caudal cell fate during neural patterning transition. J. Biol. Chem. 293, 4445-4455 (2018).
20. Moya, N., Cutts, J., Gaasterland, T., Willert, K. & Brafman, D. A. Endogenous WNT Signaling Regulates hPSC-Derived Neural Progenitor Cell Heterogeneity and Specifies Their Regional Identity. Stem Cell Rep. 3, 1015-1028 (2014).
21. Sareen, D. et al. Human induced pluripotent stem cells are a novel source of neural progenitor cells (iNPCs) that migrate and integrate in the rodent spinal cord: Human neural progenitor cells. J. Comp. Neurol. 522, 2707-2728 (2014).
22. Rowland, J. W. et al. Generation of Neural Stem Cells from Embryonic Stem Cells Using the Default Mechanism: In Vitro and In Vivo Characterization. Stem Cells Dev. 20, 1829-1845 (2011).
23. Smukler, S. R., Runciman, S. B., Xu, S. & van der Kooy, D. Embryonic stem cells assume a primitive neural stem cell fate in the absence of extrinsic influences. J. Cell Biol. 172, 79-90 (2006).
24. Bai, Q. et al. Temporal Analysis of Genome Alterations Induced by Single-Cell Passaging in Human Embryonic Stem Cells. Stem Cells Dev. 24, 653-662 (2015).
25. Garitaonandia, I. et al. Increased Risk of Genetic and Epigenetic Instability in Human Embryonic Stem Cells Associated with Specific Culture Conditions. PLOS ONE 10, e0118307 (2015).
26. Sirbu, I. O. & Duester, G. Retinoic-acid signalling in node ectoderm and posterior neural plate directs left—right patterning of somitic mesoderm. Nat. Cell Biol. 8, 271-277 (2006).
27. Torres, J., Prieto, J., Durupt, F. C., Broad, S. & Watt, F. M. Efficient Differentiation of Embryonic Stem Cells into Mesodermal Precursors by BMP, Retinoic Acid and Notch Signalling. PLoS ONE 7, (2012).
28. Mellott, D. O., Thisdelle, J. & Burke, R. D. Notch signaling patterns neurogenic ectoderm and regulates the asymmetric division of neural progenitors in sea urchin embryos. Development 144, 3602-3611 (2017).
29. Schmidt, M. H. H. et al. Epidermal growth factor-like domain 7 (EGFL7) modulates Notch signalling and affects neural stem cell renewal. Nat. Cell Biol. 11,873-880 (2009).
30. Verrier, L., Davidson, L., Gierlinski, M., Dady, A. & Storey, K. G. Neural differentiation, selection and transcriptomic profiling of human neuromesodermal progenitor-like cells in vitro. Development 145, dev166215 (2018).
31. Gouti, M. et al. In Vitro Generation of Neuromesodermal Progenitors Reveals Distinct Roles for Wnt Signalling in the Specification of Spinal Cord and Paraxial Mesoderm Identity. PLOS Biol. 12, e1001937 (2014).
32. Attardi, A. et al. Neuromesodermal progenitors are a conserved source of spinal cord with divergent growth dynamics. Development 145, dev166728 (2018).
33. Lippmann, E. S. et al. Deterministic HOX patterning in human pluripotent stem cell-derived neuroectoderm. Stem Cell Rep. 4,632-644 (2015).
34. Joyner, A. L., Liu, A. & Millet, S. Otx2, Gbx2 and Fgf8 interact to position and maintain a mid— hindbrain organizer. Curr. Opin. Cell Biol. 12,736-741 (2000).
35. Liu, J.-P., Laufer, E. & Jessell, T. M. Assigning the Positional Identity of Spinal Motor Neurons: Rostrocaudal Patterning of Hox-c Expression by FGFs, Gdf11, and Retinoids. Neuron 32,997-1012 (2001).
36. Resende, T. P. et al. Sonic hedgehog in temporal control of somite formation. Proc. Natl. Acad. Sci. U. S. A. 107,12907-12912 (2010).
37. Aulehla, A. et al. Wnt3a plays a major role in the segmentation clock controlling somitogenesis. Dev. Cell 4,395-406 (2003).
38. Delfino-Machín, M., Lunn, J. S., Breitkreuz, D. N., Akai, J. & Storey, K. G. Specification and maintenance of the spinal cord stem zone. Dev. Camb. Engl. 132,4273-4283 (2005).
39. Olivera-Martinez, I., Harada, H., Halley, P. A. & Storey, K. G. Loss of FGF-dependent mesoderm identity and rise of endogenous retinoid signalling determine cessation of body axis elongation. PLoS Biol. 10, e1001415 (2012).
40. Okada, Y., Shimazaki, T., Sobue, G. & Okano, H. Retinoic-acid-concentration-dependent acquisition of neural cell identity during in vitro differentiation of mouse embryonic stem cells. Dev. Biol. 275,124-142 (2004).
41. Irioka, T., Watanabe, K., Mizusawa, H., Mizuseki, K. & Sasai, Y. Distinct effects of caudalizing factors on regional specification of embryonic stem cell-derived neural precursors. Dev. Brain Res. 154, 63-70 (2005).
42. Nordström, U., Jessell, T. M. & Edlund, T. Progressive induction of caudal neural character by graded Wnt signaling. Nat. Neurosci. 5, 525-532 (2002).
43. Derossi, D., Williams, E. J., Green, P. J., Dunican, D. J. & Doherty, P. Stimulation of mitogenesis by a cell-permeable PI 3-kinase binding peptide. Biochem. Biophys. Res. Commun. 251, 148-152 (1998).
44. Nagoshi, N. & Okano, H. iPSC-derived neural precursor cells: potential for cell transplantation therapy in spinal cord injury. Cell. Mol. Life Sci. 75, 989-1000 (2018).
45. Ahuja, C. S. et al. Traumatic spinal cord injury. Nat. Rev. Dis. Primer 3, 1-21 (2017).
46. Nagoshi, N. et al. Human Spinal Oligodendrogenic Neural Progenitor Cells Promote Functional Recovery After Spinal Cord Injury by Axonal Remyelination and Tissue Sparing: Oligodendrogenic NPCs for Spinal Cord Injury. STEM CELLS Transl. Med. 7, 806-818 (2018).
47. Khazaei, M., Ahuja, C. S. & Fehlings, M. G. Generation of Oligodendrogenic Spinal Neural Progenitor Cells From Human Induced Pluripotent Stem Cells. Curr. Protoc. Stem Cell Biol. 42, 2D.20.1-2D.20.14 (2017).
Baptiste, D. C., and Fehlings, M. G. (2006). Pharmacological approaches to repair the injured spinal cord. J. Neurotrauma 23, 318-334.
Bonner, J. F., and Steward, O. (2015). Repair of spinal cord injury with neuronal relays: From fetal grafts to neural stem cells. Brain Res. 1619, 115-123.
Bresnahan, J. C., Beattie, M. S., Todd III, F. D., and Noyes, D. H. (1987). A behavioral and anatomical analysis of spinal cord injury produced by a feedback-controlled impaction device. Exp. Neurol. 95, 548-570.
Chamankhah, M., Eftekharpour, E., Karimi-Abdolrezaee, S., Boutros, P. C., San-Marina, S., and Fehlings, M. G. (2013). Genome-wide gene expression profiling of stress response in a spinal cord clip compression injury model. BMC Genomics 14, 583.
Chang, D.-J., Lee, N., Park, I.-H., Choi, C., Jeon, I., Kwon, J., Oh, S.-H., Shin, D. A., Do, J. T., Lee, D. R., et al. (2013). Therapeutic potential of human induced pluripotent stem cells in experimental stroke. Cell Transplant. 22, 1427-1440.
Chen, J., Leong, S.-Y., and Schachner, M. (2005). Differential expression of cell fate determinants in neurons and glial cells of adult mouse spinal cord after compression injury. Eur. J. Neurosci. 22, 1895-1906.
Chen, Q., Zhang, M., Li, Y., Xu, D., Wang, Y., Song, A., Zhu, B., Huang, Y., and Zheng, J. C. (2015). CXCR7 Mediates Neural Progenitor Cells Migration to CXCL12 Independent of CXCR4. Stem Cells Dayt. Ohio 33, 2574-2585.
Dai, H., Macarthur, L., McAtee, M., Hockenbury, N., Das, P., and Bregman, B. S. (2011). Delayed rehabilitation with task-specific therapies improves forelimb function after a cervical spinal cord injury. Restor. Neurol. Neurosci. 29, 91-103.
De Biase, A., Knoblach, S. M., Di Giovanni, S., Fan, C., Molon, A., Hoffman, E. P., and Faden, A. I. (2005). Gene expression profiling of experimental traumatic spinal cord injury as a function of distance from impact site and injury severity. Physiol. Genomics 22, 368-381.
Dusart, I., and Schwab, M. E. (1994). Secondary Cell Death and the Inflammatory Reaction After Dorsal Hemisection of the Rat Spinal Cord. Eur. J. Neurosci. 6, 712-724.
Ferrari, D., Zalfa, C., Nodari, L. R., Gelati, M., Carlessi, L., Delia, D., Vescovi, A. L., and De Filippis, L. (2012). Differential pathotropism of non-immortalized and immortalized human neural stem cell lines in a focal demyelination model. Cell. Mol. Life Sci. CMLS 69, 1193-1210.
Filippo, T. R. M., Galindo, L. T., Barnabe, G. F., Ariza, C. B., Mello, L. E., Juliano, M. A., Juliano, L., and Porcionatto, M. A. (2013). CXCL12 N-terminal end is sufficient to induce chemotaxis and proliferation of neural stem/progenitor cells. Stem Cell Res. 11, 913-925.
Franklin, R. J. M., and Hinks, G. L. (1999). Understanding CNS remyelination: Clues from developmental and regeneration biology. J. Neurosci. Res. 58, 207-213.
Furlan, J. C., Noonan, V., Singh, A., and Fehlings, M. G. (2011). Assessment of impairment in patients with acute traumatic spinal cord injury: a systematic review of the literature. J. Neurotrauma 28, 1445-1477.
Garcia-Alias, G., Barkhuysen, S., Buckle, M., and Fawcett, J. W. (2009). Chondroitinase ABC treatment opens a window of opportunity for task-specific rehabilitation. Nat. Neurosci. 12, 1145-1151.
Gifford, W. D., Hayashi, M., Sternfeld, M., Tsai, J., Alaynick, W. A., and Pfaff, S. L. (2013). Chapter 7—Spinal Cord Patterning. In Patterning and Cell Type Specification in the Developing CNS and PNS, J. L. R. Rubenstein, and P. Rakic, eds. (Oxford: Academic Press), pp. 131-149.
Grandbarbe, L., Bouissac, J., Rand, M., Angelis, M. H. de, Artavanis-Tsakonas, S., and Mohier, E. (2003). Delta-Notch signaling controls the generation of neurons/glia from neural stem cells in a stepwise process. Development 130, 1391-1402.
Gray, E. G. (1959). Electron microscopy of synaptic contacts on dendrite spines of the cerebral cortex. Nature 183, 1592-1593.
Hofstetter, C. P., Holmstrom, N. A. V., Lilja, J. A., Schweinhardt, P., Hao, J., Spenger, C., Wiesenfeld-Hallin, Z., Kurpad, S. N., Frisen, J., and Olson, L. (2005). Allodynia limits the usefulness of intraspinal neural stem cell grafts; directed differentiation improves outcome. Nat. Neurosci. 8, 346-353.
Hussein, S. M., Batada, N. N., Vuoristo, S., Ching, R. W., Autio, R., Närvä, E., Ng, S., Sourour, M., Hämäläinen, R., Olsson, C., et al. (2011). Copy number variation and selection during reprogramming to pluripotency. Nature 471, 58-62.
Imitola, J., Raddassi, K., Park, K. I., Mueller, F.-J., Nieto, M., Teng, Y. D., Frenkel, D., Li, J., Sidman, R. L., Walsh, C. A., et al. (2004). Directed migration of neural stem cells to sites of CNS injury by the stromal cell-derived factor 1a/CXC chemokine receptor 4 pathway. Proc. Natl. Acad. Sci. 101, 18117-18122.
Karimi-Abdolrezaee, S., Eftekharpour, E., Wang, J., Morshead, C. M., and Fehlings, M. G. (2006). Delayed transplantation of adult neural precursor cells promotes remyelination and functional neurological recovery after spinal cord injury. J. Neurosci. Off. J. Soc. Neurosci. 26, 3377-3389.
Khazaei, M., Ahuja, C. S., and Fehlings, M. G. (2017). Induced Pluripotent Stem Cells for Traumatic Spinal Cord Injury. Front. Cell Dev. Biol. 4.
Li, L., Velumian, A. A., Samoilova, M., and Fehlings, M. G. (2016). A Novel Approach for Studying the Physiology and Pathophysiology of Myelinated and Non-Myelinated Axons in the CNS White Matter. PLOS ONE 11, e0165637.
Li, Y. Q., Guo, Y. P., Jay, V., Stewart, P. A., and Wong, C. S. (1996). Time course of radiation-induced apoptosis in the adult rat spinal cord. Radiother. Oncol. 39, 35-42.
Lippmann, E. S., Williams, C. E., Ruhl, D. A., Estevez-Silva, M. C., Chapman, E. R., Coon, J. J., and Ashton, R. S. (2015). Deterministic HOX patterning in human pluripotent stem cell-derived neuroectoderm. Stem Cell Rep. 4, 632-644.
Liu, X. Z., Xu, X. M., Hu, R., Du, C., Zhang, S. X., McDonald, J. W., Dong, H. X., Wu, Y. J., Fan, G. S., Jacquin, M. F., et al. (1997). Neuronal and Glial Apoptosis after Traumatic Spinal Cord Injury. J. Neurosci. 17, 5395-5406.
Lu, P., Wang, Y., Graham, L., McHale, K., Gao, M., Wu, D., Brock, J., Blesch, A., Rosenzweig, E. S., Havton, L. A., et al. (2012). Long-distance growth and connectivity of neural stem cells after severe spinal cord injury. Cell 150, 1264-1273.
Lu, P., Woodruff, G., Wang, Y., Graham, L., Hunt, M., Wu, D., Boehle, E., Ahmad, R., Poplawski, G., Brock, J., et al. (2014). Long-Distance Axonal Growth from Human Induced Pluripotent Stem Cells after Spinal Cord Injury. Neuron 83, 789-796.
Miyagi, M., Ishikawa, T., Kamoda, H., Orita, S., Kuniyoshi, K., Ochiai, N., Kishida, S., Nakamura, J., Eguchi, Y., Arai, G., et al. (2011). Assessment of gait in a rat model of myofascial inflammation using the CatWalk system. Spine 36, 1760-1764.
Nagoshi, N., Khazaei, M., Ahlfors, J.-E., Ahuja, C. S., Nori, S., Wang, J., Shibata, S., and Fehlings, M. G. (2018). Human Spinal Oligodendrogenic Neural Progenitor Cells Promote Functional Recovery After Spinal Cord Injury by Axonal Remyelination and Tissue Sparing. Stem Cells Transl. Med. Namihira, M., Kohyama, J., Semi, K., Sanosaka, T., Deneen, B., Taga, T., and Nakashima, K. (2009). Committed neuronal precursors confer astrocytic potential on residual neural precursor cells. Dev. Cell 16, 245-255.
Nashmi, R., and Fehlings, M. G. (2001). Changes in axonal physiology and morphology after chronic compressive injury of the rat thoracic spinal cord. Neuroscience 104, 235-251.
Nguyen, D. H., Cho, N., Satkunendrarajah, K., Austin, J. W., Wang, J., and Fehlings, M. G. (2012). Immunoglobulin G (IgG) attenuates neuroinflammation and improves neurobehavioral recovery after cervical spinal cord injury. J. Neuroinflammation 9.
Pataky, D. M., Borisoff, J. F., Fernandes, K. J., Tetzlaff, W., and Steeves, J. D. (2000). Fibroblast growth factor treatment produces differential effects on survival and neurite outgrowth from identified bulbospinal neurons in vitro. Exp. Neurol. 163, 357-372.
Payne, S. L., Anandakumaran, P. N., Varga, B. V., Morshead, C. M., Nagy, A., and Shoichet, M. S. (2018). In Vitro Maturation of Human iPSC-Derived Neuroepithelial Cells Influences Transplant Survival in the Stroke-Injured Rat Brain. Tissue Eng. Part A 24, 351-360.
Rowland, J. W., Hawryluk, G. W. J., Kwon, B., and Fehlings, M. G. (2008). Current status of acute spinal cord injury pathophysiology and emerging therapies: promise on the horizon. Neurosurg. Focus 25, E2.
Ruff, C. A., Wilcox, J. T., and Fehlings, M. G. (2012). Cell-based transplantation strategies to promote plasticity following spinal cord injury. Exp. Neurol. 235, 78-90.
Salewski, R. P., Buttigieg, J., Mitchell, R. A., van der Kooy, D., Nagy, A., and Fehlings, M. G. (2013). The generation of definitive neural stem cells from PiggyBac transposon-induced pluripotent stem cells can be enhanced by induction of the NOTCH signaling pathway. Stem Cells Dev. 22, 383-396.
Salewski, R. P., Mitchell, R. A., Li, L., Shen, C., Milekovskaia, M., Nagy, A., and Fehlings, M. G. (2015). Transplantation of Induced Pluripotent Stem Cell-Derived Neural Stem Cells Mediate Functional Recovery Following Thoracic Spinal Cord Injury Through Remyelination of Axons. Stem Cells Transl. Med. 4, 743-754.
Sansom, S. N., Griffiths, D. S., Faedo, A., Kleinjan, D.-J., Ruan, Y., Smith, J., Heyningen, V. van, Rubenstein, J. L., and Livesey, F. J. (2009). The Level of the Transcription Factor Pax6 Is Essential for Controlling the Balance between Neural Stem Cell Self-Renewal and Neurogenesis. PLOS Genet e1000511.
Sekhon, L. H., and Fehlings, M. G. (2001). Epidemiology, demographics, and pathophysiology of acute spinal cord injury. Spine 26, S2-12.
Silbereis, J. C., Pochareddy, S., Zhu, Y., Li, M., and Sestan, N. (2016). The Cellular and Molecular Landscapes of the Developing Human Central Nervous System. Neuron 89,248-268.
Soubeyrand, M., Badner, A., Vawda, R., Chung, Y. S., and Fehlings, M. G. (2014). Very high resolution ultrasound imaging for real-time quantitative visualization of vascular disruption after spinal cord injury. J. Neurotrauma 31,1767-1775.
Stangel, M., and Hartung, H.-P. (2002). Remyelinating strategies for the treatment of multiple sclerosis. Prog. Neurobiol. 68,361-376.
Tanigaki, K., Nogaki, F., Takahashi, J., Tashiro, K., Kurooka, H., and Honjo, T. (2001). Notch1 and Notch3 instructively restrict bFGF-responsive multipotent neural progenitor cells to an astroglial fate. Neuron 29,45-55.
Tator, C. H., Duncan, E. G., Edmonds, V. E., Lapczak, L. I., and Andrews, D. F. (1993). Changes in epidemiology of acute spinal cord injury from 1947 to 1981. Surg. Neurol. 40,207-215.
Varga, B. V., Faiz, M., Yang, H., Gao, S., Linderoth, E., Zhen, M., Hussein, S. M., and Nagy, A. Signal requirement for forebrain potential of transplantable human neuroepithelial stem cells. Cell Stem Cell

In revision, Submitted.
Wilcox, J. T., Satkunendrarajah, K., Zuccato, J. A., Nassiri, F., and Fehlings, M. G. (2014). Neural precursor cell transplantation enhances functional recovery and reduces astrogliosis in bilateral compressive/contusive cervical spinal cord injury. Stem Cells Transl. Med. 3,1148-1159.
Wilcox, J. T., Satkunendrarajah, K., Nasirzadeh, Y., Laliberte, A. M., Lip, A., Cadotte, D. W., Foltz, W. D., and Fehlings, M. G. (2017). Generating level-dependent models of cervical and thoracic spinal cord injury: Exploring the interplay of neuroanatomy, physiology, and function. Neurobiol. Dis. 105, 194-212.

1-51. (canceled)

52. A method of producing spinal identity neural progenitor cells (spNPCs), the method comprising:
a. passaging posteriorized NPCs and incubating the posteriorized NPCs in culture media supplemented with a RAR agonist, to produce caudalized NPCs expressing reduced levels of at least one of Gbx2, Otx2 and FoxG1 levels compared to posteriorized NPCs;
b. passaging the caudalized NPCs in suitable culture media supplemented with a RAR agonist; and
c. passaging the caudalized NPCs of step b) in suitable culture media supplemented with a FGF2 agonist, an EGF receptor agonist, and 740Y-P or a synthetic agonist of 740Y-P, until the identity of the NPCs are stabilized as spN PCs.

53. The method of claim 52, wherein the posteriorized NPCs are obtained by dissociating unpatterned NPCs primed to stay in an ectodermal cell fate and incubating the primed unpatterned NPCs in culture media supplemented with a FGF2 agonist and a FGF8 agonist, to produce posteriorized NPCs expressing higher levels of at least one Hox gene, and lower levels of at least one of the brain markers such as Gbx2, Otx2 and FoxG1 compared to unpatterned NPCs.

54. The method of claim 53, wherein the primed unpatterned NPCs are obtained by a method comprising:
a. obtaining unpatterned NPCs, the unpatterned NPCs expressing neuroectodermal markers including Pax6 and Sox1;
b. priming the unpatterned NPCs of step a, the method comprising adding EGF-L7 agonist to culture media comprising the unpatterned NPCs of step a.

55. The method of claim 54, wherein priming the unpatterned NPCs further comprises adding a Notch signaling activator to the culture media.

56. The method of claim 53, wherein the unpatterned NPCs are obtained from induced pluripotent stem cells (iPSCs), the method comprising:
a. passaging the iPSCs and incubating said cells in iPSC culture media for about 2 days to about 4 days;
b. culturing the iPSCs in iPSC culture media without a FGF2 agonist, for about 4 days, wherein a BMP inhibitor or dual SMAD inhibitors are added to the culture media between about day 2 to about day 4;
c. culturing the iPSCs in NIM without FGF2 agonist, for about 2 days to produce embryoid bodies (EB); and
d. culturing the EBs of step c) in NIM with a FGF2 agonist, for about 7 to about 11 days to produce neural rosettes, wherein the BMP inhibitor or dual SMAD inhibitors are removed from the media on about day 2, to produce unpatterned NPCs.

57. The method of claim 56, wherein the iPSC culture media of step a) comprises a BMP inhibitor, TGFβ inhibitor, FGF2 agonist, and Wnt inhibitor.

58. The method of claim 52, wherein a ROCK inhibitor is added to the culture media on day 1 after each or at least one passage.

59. The method of claim 52, wherein the RAR agonist is RA or a RA synthetic analog such as EC23.

60. The method of claim 52, wherein the posteriorized NPCs are incubated in culture media supplemented with a Wnt signaling activator in addition to the RAR agonist.

61. The method of claim 60, wherein the Wnt signaling activator is Wnt3a, AZD2858, Wnt agonist 1, CP21R7 (CP21), Wnt or BML-284 hydrochloride.

62. The method of claim 52, wherein the FGF2 agonist is FGF2 or SUN11602.

63. The method of claim 52, wherein the EGF receptor agonist is EGF or NSC228155.

64. An isolated cell population comprising spNPCs produced according to a method comprising:
a. passaging posteriorized NPCs and incubating the posteriorized NPCs in culture media supplemented with a RAR agonist, to produce caudalized NPCs expressing reduced levels of at least one of Gbx2, Otx2 and FoxG1 levels compared to posteriorized NPCs;
b. passaging the caudalized NPCs in suitable culture media supplemented with a RAR agonist; and
c. passaging the caudalized NPCs of step b) in suitable culture media supplemented with a FGF2 agonist, an EGF receptor agonist, and 740Y-P or a synthetic agonist of 740Y-P, until the identity of the NPCs are stabilized as spN PCs.

65. A method of treating a subject with a spinal cord injury or a neurodegenerative disease, comprising administering the isolated cell population of claim 64 to the subject.

66. A method of generating human neural stem/progenitor cells or neural precursor cell with spinal cord identity (spNPCs) comprising the following steps:
a. suspending pluripotent stem cells in a culture media containing a TGFβ inhibitor, FGF2 agonist, Wnt inhibitor, and BMP inhibitor;
b. subjecting the cells obtained in step (a) to suspension culture in a culture media containing a Wnt inhibitor and a BMP inhibitor;
c. forming an embryoid body by contacting the pluripotent human cell with an essentially serum free medium;
d. culturing the embryoid body to form rosettes and neural tube-like structure and neuroectodermal cells;
e. priming the neuroectodermal cells to stay in the ectodermal cell fate by using EGF-L7 or its agonist(s);
f. posteriorizing the cells primed in e) in high concentration of FGF2 and FGF8;
g. passaging the cells posteriorized in f) and caudalizing the posteriorized cells in culture media supplemented with a RA or a RA synthetic analog and a Wnt agonist such as AZD2858, Wnt agonist 1, CP21R7 (CP21) or Wnt;
h. passaging the cells caudalized in g) in suitable culture media supplemented with a RAR agonist;
i. passaging the caudalized cells of step h) in suitable culture media supplemented with a FGF2 agonist, an EGF receptor agonist, and 740Y-P or a synthetic agonist of 740Y-P, until the identity of the cells are stabilized as spNPCs; and
j. inducing proliferation capacity of spinal NPCs from the cells generated in step g) by dual activation of PI 3-kinase-Akt pathway and FGF pathway.

67. The method of claim 66, wherein 740Y-P is used for the dual activation of PI 3-kinase-Akt pathway and FGF pathway.

68. A cell culture composition for use in a step of deriving the spinal NPCs of claim 66 in vitro from pluripotent stem cells, wherein the spinal NPCs express one or more detectable markers for Sox2, Pax6, Nestin or vimentin, and the spinal NPCs have the capacity to differentiate into cells of a neural lineage.

69. The cell culture composition of claim 68 for use to differentiate human pluripotent stem cells into spinal-NPCs, wherein the base media for each step is described in Table 1.

70. An isolated population of human pluripotent stem cell derived spinal neural stem/progenitor cells (spNPCs) produced according to the method of claim 66.

71. A method of treating a subject with a spinal cord injury or neurodegenerative disorder, comprising administering the isolated population of spNPCs of claim 70 to the subject.
